SEQUENCE LISTING (8 sequences)

SEQ ID NO: 1 - DNA, 1050 bp, Helicobacter pylori
  CDS (1)..(1050)
atgagcgtaaatgcacccaaacgcatgcgtattttattgcgtttgcctaattggttaggcgatggggtgatggcaagttcgcttttttacacccttaaacaccactaccctaacgcgcattttatcttagtgggcccaaccattacttgcgaacttttcaaaaaagatgaaaaaatagaagccgtttttatagacaacaccaaaaaatcctttttcaggctgctagccattcacaaactcgctcaaaaaatagggcgttgcgatatagcgatcactttaaacaaccatttctattccgcttttttgctctatgcgacaaaaacgcccgttcgcatcggttttgctcaattttttcgttctttgtttctcagccatgcgatcgctcctgcccctaaagagtatcaccaagtggaaaagtattgctttttattttcgcaatttttagaaaaagaattggatcaaaaaagcgttttacccttaaaactggcctttaacctccccactcacaccccaaacacccctaaaaaaatcggctttaaccctagcgcaagctatgggagtgctaaaagatggccagcttcttattacgctgaagtttctgctgttttgttagaaaaagggcatgaaatttatttttttggggctaaagaagacgctatcgtttctgaagaaattttaaaactcatcaaaggctcattaaaaaacccctcattgttccataacgcttacaatctgtgcgggaaaacaagcattgaagaattgatagagcgcatcgctgttttagatttattcatcactaacgatagcggccctatgcatgtggctgctagcatgcaaacccccttaatcgctctttttggccccactgatgaaaaagagactcgcccctataaagctcaaaaaacgatcgtattgaaccaccatttaagctgtgcgccttgcaagaaacgagtttgccctttaaagaatgcaaaaaaccatttgtgcatgaaatctatcacgccccttgaagtcctagaagccgctcacactcttttagaagagccttaa

SEQ ID NO: 2 - protein, 349 aa, Helicobacter pylori
MSVNAPKRMRILLRLPNWLGDGVMASSLFYTLKHHYPNAHFILVGPTITCELFKKDEKIEAVFIDNTKKSFFRLLAIHKLAQKIGRCDIAITLNNHFYSAFLLYATKTPVRIGFAQFFRSLFLSHAIAPAPKEYHQVEKYCFLFSQFLEKELDQKSVLPLKLAFNLPTHTPNTPKKIGFNPSASYGSAKRWPASYYAEVSAVLLEKGHEIYFFGAKEDAIVSEEILKLIKGSLKNPSLFHNAYNLCGKTSIEELIERIAVLDLFITNDSGPMHVAASMQTPLIALFGPTDEKETRPYKAQKTIVLNHHLSCAPCKKRVCPLKNAKNHLCMKSITPLEVLEAAHTLLEEP

SEQ ID NO: 3 - DNA, 1072 bp, Helicobacter pylori
gttaagcccattcctttgacgatagagtttgcatgaaaatagcgattgtcaggctttcagcgcttggggatattatcgtgagcgcggtgtttttggcggtgattaaagagtgtctgcctaacgcccaaatagaatggttcgtggatgaaagatttagtgcgattttagagcattccccctatattgataaattacaccccatcgctttaaaaagtgcactcaaaaccttgaatcctttgaagattttcaaactttttaaatctttaagggcttatgaatacgatataatcattgacatgcaaggcctagtcaaatccgctctcatcacgcaaatgttgaaagcccctaaaaaagtcggctttgattacgcttcggctagagagggtttgagcatgtttttttactcgcaaaaagtttctatcgcttatgatgagcctgttttaaagcgcaatttcacgctcctttctcatgccctaaacttgccccaaaaagaaatttcaaaagaaatttcagagagcttaagctctagggctaaagcgttttcttaccagccttctccaaaaattgatgcgttaaatttgaataagaataagccaaaaatcctttttattttagaaacttctaaaatcaataaaacttaccccatagagcgttttaaagaattagcgttaattttagaaaattttcaaatttgcttgttatggcatgctgatgaatataaagccactacgctttatcacgctttaaaacaccaacgcgatgtgttattgctccccaaactcactttaaacgaggttaaggcgttgctctttaaaatggatttgattattgggggcgatacgggcatcacgcatttagcatgggcgttgcaaaaacccagcatcaccctttatggcaacacgcccatggagcgttttaaattagaaagcccgatcaatgtttcgctcaccggtaattcaaacgccaactaccataaaaaggatttttctatccaaaatatagagcctaaaaaaattaaagaatgcgttttaaacatcttaaaggaaaaagaatgacttacaaagaacgactc

SEQ ID NO: 4 - protein, 340 aa, Helicobacter pylori
MKIAIVRLSALGDIIVSAVFLAVIKECLPNAQIEWFVDERFSAILEHSPYIDKLHPIALKSALKTLNPLKIFKLFKSLRAYEYDIIIDMQGLVKSALITQMLKAPKKVGFDYASAREGLSMFFYSQKVSIAYDEPVLKRNFTLLSHALNLPQKEISKEISESLSSRAKAFSYQPSPKIDALNLNKNKPKILFILETSKINKTYPIERFKELALILENFQICLLWHADEYKATTLYHALKHQRDVLLLPKLTLNEVKALLFKMDLIIGGDTGITHLAWALQKPSITLYGNTPMERFKLESPINVSLTGNSNANYHKKDFSIQNIEPKKIKECVLNILKEKE

SEQ ID NO: 5 - DNA, 30 bp, Artificial Sequence
  Primer
cgggatcccggtctttaaacccgctcaaca

SEQ ID NO: 6 - DNA, 29 bp, Artificial Sequence
  Primer
cgggatccccgctcttctcacgcctttaa

SEQ ID NO: 7 - DNA, 21 bp, Artificial Sequence
  Primer
atcatttaaaatacacgctag

SEQ ID NO: 8 - DNA, 18 bp, Artificial Sequence
  Primer
cctaccgcttcccaatag

(19) United States
(12) Patent Application Publication
(10) Pub. No.: US 2004/0115669 A1
(43) Pub. Date: Jun. 17, 2004

(54) Helicobacter pylori heptosyl transferase polypeptides

(76) Inventors: Koji Hiratsuka, Stittsville (CA); Susan M Logan, Ottawa (CA)

Correspondence Address: George A Seaby, 603 - 800 Wellington Street, Ottawa, ON, K1R 6K7, CA

(21) Appl. No.: 10/451,686

(22) PCT Filed: Jun. 28, 2001
(86) PCT No.: PCT/CA01/00970

(51) Int. Cl. C12Q 1/68; C07H 21/04; C12N 9/10

(52) U.S. Cl. 435/6; 435/69.1; 435/193; 435/320.1; 435/325; 530/388.26; 536/23.2

(57) ABSTRACT

This invention relates to newly identified polynucleotides and polypeptides, and their production and uses, as well as their variants, agonists and antagonists, and their uses. In particular, the invention relates to novel heptosyltransferase polynucleotides and polypeptides.

### FIELD OF THE INVENTION

This invention relates to newly identified polynucleotides and polypeptides, and their production and uses, as well as their variants, agonists and antagonists, and their uses. In particular, the invention relates to novel heptosylbansferase polynucleotides and polypeptides.

### BACKGROUND OF THE INVENTION

Helicobacter pylori is a spiral Gram negative bacterium which colonizes the human stomach. It is estimated that up to 50% of the human population is infected with H. pylori (Dunn et al., 1997). Thus, H. pylori remains one of the most prevalent bacterial pathogens worldwide.

Infection by H. pylori is associated with chronic superficial and active gastritis (Blaser, 1990), which may eventually develop into peptic ulcers (Blaser, 1995; Graham, 1991). Furthermore, prolonged infection by H. pylori can lead to the development of gastric carcinoma and mucosa-associated lymphoid tissue (MALI) lymphoma (Dunn, et al, 1997; Parsonnet, et al, 1994). H. pylori has been declared a human carcinogen by the International Agency for Cancer Research. Many research initiatives worldwide are aimed at determining the reasons why H. pylori produces such a variety of pathogenic outcomes.

Genetic variations in both the host and pathogen likely explain much of the clinical variation. Genomic mapping of several H. pylori strains showed a variation in the arrangement of several genetic markers (Jiang et al, 1996). This variability was also observed in gene organization, gene content and nucteotide sequence between the genome sequences of two H. pylori strains that have been determined and annotated (Tombs, et al., 1997; Alm, et al., 1999). Several factors associated with pathogenicity have been identified, including urease, vacuolating cytotoxin (VAC), cytotoxin associated gene (CAG); various adhesins, iron-binding proteins, catalase, superoxide dismutase and lipopolysaccharide (LPS)(for review see Dunn et al., 1997).

The LPS of H. pylori may play several roles in pathogenesis. In particular, H. pylori LPS has been implicated in causing abnormal acid secretion and in inducing apoptosis of epithelial cells and gastritis in mice (Piotrowski, et al., 1997a; Sakagami, et al., 1997; Kidd, et al., 1997; Piotrowski, et al., 1997b; Ootsubo, et al., 1997; Okumura; et al., 1998). H. pylori LPS may also be involved in triggering inflammatory response. Additionally, some strains of H. pylori express O-antigen polysaccharide chains which mimic Lewis blood group antigens (Aspinall, et al., 1997; Monteiro et al., 1998b) which are naturally expressed in the human gastric mucosa. Such antigenic mimicry may play a role in evasion of the host immune system. Alternatively, this mimicry may give rise to pathogenic autoimmune antibodies by the host (Appelmelk, et al., 1997). The exposure of LPS at the bacterial cell surface would make it an obvious putative colonization factor. Recently Edwards et al. (2000) showed that the O-chain polysaccharide found in the LPS of many strains of H. pylori may be involved in the adhesion of the pathogen to gastric epithelial cells. Similarly, Logan et al. (2000) have shown O-antigen to be an important H. pylori factor for the colonization of the murine stomach.

### SUMMARY OF THE INVENTION

Applicants have identified, cloned and characterized genes involved in assembly of the core polysaccharide of the LPS molecule. The genes encode heptosyltransferases obtainable from Helicobacter that are responsible for adding heptose to the core LPS structure. One of the heptosyltransferases has been identified as an ADP heptose-LPS-heptosyltransferase II (WaaF) that adds L-glycero-D-manno-heptose to the inner core of the LPS (SEQ ID NO 1, and 2). A heptosyltransferase I (WaaC) has also been identified (SEQ ID NO 3, and 4).

Polypeptides of the invention are referred to herein as “HepT” or “HepT polypeptides” and polynucleotides encoding polypeptides of the invention are referred to herein as “hepT gene” or “hepT”

Broadly stated the present invention contemplates isolated polynucleotides encoding HepT polypeptides of the invention, including mRNAs, DNAs, cDNAs, genomic DNAs, PNAs, as well as antisense analogs and biologically, diagnostically, prophylactically, clinically or therapeutically useful variants or fragments thereof, and compositions comprising same.

In particular, the present invention contemplates an isolated polynucleotide comprising a sequence that comprises at least 18 nucleotides and hybridizes under stringent conditions to the complementary nucleic acid sequence of SEQ. ID. NO. 1 or 3 or a degenerate form thereof. In an embodiment the polynucleotide comprises a region encoding a HepT polypeptide comprising a sequence set out in SEQ ID NO: 1 or 3 which includes a full length polynucleotide or a variant thereof. In a preferred embodiment the polynucleotide encodes a polynucleotide designated herein as HP0279 or HP1191.

The polynucleotides of the invention permit identification of untranslated nucleic acid sequences or regulatory sequences which specifically promote expression of genes operatively linked to the promoter regions. The invention therefore contemplates a polynucleotide encoding a regulatory sequence of a polynucleotide of the invention such as a promoter sequence, preferably a regulatory sequence of a hepT gene.

The polynucleotides encoding a mature polypeptide of the invention may include only the coding sequence for the mature polypeptide; the coding sequence for the mature polypeptide and additional coding sequences (e.g. leader or secretory sequences, proprotein sequences); the coding sequence for the mature polypeptide (and optionally additional coding sequences) and non-coding sequence, such as introns or non-coding sequences 5′ and/or 3′ of the coding sequence of the mature polypeptide.

The polynucleotides of the invention may be inserted into an appropriate expression vector, and the vector may contain the necessary elements for the transcription and translation of an inserted coding sequence. Accordingly, recombinant expression vectors may be constructed which comprise a polynucleotide of the invention, and where appropriate one or more transcription and translation elements linked to the polynucleotide.

Vectors are contemplated within the scope of the invention which comprise regulatory sequences of the invention, as well as chimeric gene constructs wherein a regulatory sequence of the invention is operably linked to a polynucleotide sequence encoding a heterologous protein (i.e. a protein not naturally expressed in the host cell), and a transcription termination signal.

A vector can be used to transform host cells to express a polypeptide of the invention, or a heterologous protein. Therefore, the invention further provides host cells containing a vector of the invention.

The invention also contemplates an isolated HepT polypeptide encoded by a polynucleotide of the invention. In an embodiment, the invention provides a HepT from Helicobacter comprising the amino acid sequence of SEQ ID NO: 2 or 4 or a variant thereof. Further embodiments of the invention provide biologically, diagnostically, prophylactically, clinically or therapeutically useful variants thereof and compositions comprising a polypeptide of the invention.

Among the embodiments of the invention are variants of a polypeptide of the invention encoded by naturally occurring alleles of a hepT gene.

Polypeptides of the invention may be obtained as an isolate from natural cell sources, but they are preferably produced by recombinant procedures. In one aspect the invention provides a method for preparing a polypeptide of the invention utilizing an isolated polynucleotide of the invention. In an embodiment a method for preparing a HepT polypeptide is provided comprising:

(a) transferring a recombinant expression vector of the invention having a polynucleotide sequence encoding a HepT polypeptide, into a host cell;

(b) selecting transformed host cells from untransformed host cells;

(c) culturing a selected transformed host cell under conditions which allow expression of the HepT polypeptide; and

(d) isolating the HepT polypeptide.

The invention further broadly contemplates a recombinant HepT obtained using a method of the invention.

A polypeptide of the invention may be conjugated with other molecules, such as proteins, to prepare fusion proteins or chimeric proteins. This may be accomplished, for example, by the synthesis of N-terminal or C-terminal fusion proteins.

The invention further contemplates antibodies having specificity against an epitope of a polypeptide of the invention. Antibodies may be labeled with a detectable substance and used to detect polypeptides of the invention in biological samples, tissues, and cells.

The invention also permits the construction of nucleotide probes which are unique to the polynucleotides of the invention or to polypeptides of the invention. Therefore, the invention also relates to a probe comprising a sequence encoding a polypeptide of the invention, or a part thereof. The probe may be labeled, for example, with a detectable substance and it may be used to select from a mixture of nucleotide sequences a polynucleotide of the invention including polynucleotides encoding a polypeptide which displays one or more of the properties of a polypeptide of the invention.

In accordance with an aspect of the invention there is provided a method of, and products for, diagnosing and monitoring diseases by determining the presence of polynucleotides and polypeptides of the invention.

Still further the invention provides a method for evaluating a test compound or agent for its ability to modulate the activity of a polypeptide or polynucleotide of the invention. For example a substance which inhibits or enhances the catalytic activity of a polypeptide of the invention may be evaluated. “Modulate” refers to a change or an alteration in the biological activity of a polypeptide of the invention. Modulation may be an increase or a decrease in activity, a change in characteristics, or any other change in the biological, functional, or immunological properties of the polypeptide.

In an embodiment, the invention provides methods for identifing compounds which bind to or otherwise interact with and inhibit or activate an activity of a polypeptide or polynucleotide of the invention comprising:

(a) contacting a polypeptide or polynucleotide of the invention with a test compound under conditions to permit binding to or other interaction between the test compound and the polypeptide or polynucleotide to assess the binding to or other interaction with the test compound, wherein the binding or interaction is associated with a second component capable of providing a detectable signal in response to the binding or interaction of the polypeptide or polynucleotide with the test compound; and

(b) determining whether the test compound binds to or interacts with and activates or inhibits an activity of the polypeptide or polynucleotide by detecting the presence or absence of a signal generated from the binding or interaction of the test compound with the polypeptide or polynucleotide.

Compounds which modulate the biological activity of a polypeptide of the invention may also be identified using the methods of the invention by comparing the pattern and level of expression of a polynucleotide or polypeptide of the invention in cells and organisms, in the presence, and in the absence of the compounds.

Methods are also contemplated that identify compounds or substances (e.g. polypeptides) which bind to regulatory sequences (e.g. promoter sequences, enhancer sequences, negative modulator sequences).

Still another aspect of the invention provides a method of conducting a drug discovery business comprising:

(a) providing one or more systems or methods for identifying modulators of a polypeptide or polynucleotide of the invention;

(b) conducting therapeutic profiling of modulators identified in step (a), or further analogs thereof, for efficacy and toxicity in animals; and

(c) formulating a pharmaceutical composition including one or more modulators identified in step (b) as having an acceptable therapeutic profile.

In certain embodiments, the subject method may also include a step of establishing a distribution system for distributing the pharmaceutical composition for sale, and may optionally include establishing a sales group for marketing the pharmaceutical composition.

In yet another aspect of the invention, a method of conducting a target discovery business is provided comprising:

(a) providing one or more systems or methods for identifying modulators of a polypeptide or polynucleotide of the invention;

(b) optionally conducting therapeutic profiling of modulators identified in (a) for efficacy and toxicity in animals; and

(c) licensing to a third party the rights for further drug development and/or sales for modulators identified in step (a), or analogs thereof.

The substances and compounds identified using the methods of the invention, antibodies, and antisense polynucleotides may be used to modulate the biological activity of a polypeptide or polynucleotide of the invention, and they may be used in the prevention and treatment of disease. In an aspect of the invention the substances and compounds are inhibitors of polypeptides of the invention that are useful as antibacterial agents.

In accordance with an aspect of the invention there are provided agonists and antagonists of a HepT polypeptide, preferably bacteriostatic or bacteriocidal agonists or antagonists.

Accordingly, the polynucleotides and polypeptides of the invention, antibodies and substances and compounds may be formulated into compositions for administration to a cell or to a multicellular organism. Therefore, the present invention also relates to a composition comprising one or more of a polynucleotide or polypeptide of the invention, antibody or a substance or compound identified using the methods of the invention, and a pharmaceutically acceptable carrier, excipient or diluent. A method for treating or preventing a disease is also provided comprising administering to a patient in need thereof, a composition of the invention.

In accordance with certain embodiments of the invention, there are provided products, compositions and methods for assessing HepT expression, treating disease, assaying genetic variation, and administering a polypeptide or polynucleotide of the invention to an organism to raise an immunological response against a bacteria.

Having provided novel HepT polypeptides, and polynucleotides encoding same, the invention accordingly further provides methods for preparing oligosaccbarides e.g. two or more saccharides. In specific embodiments, the invention relates to a method for preparing an oligosaccharide comprising contacting a reaction mixture comprising an activated donor molecule, and an acceptor in the presence of a polypeptide of the invention.

In accordance with a further aspect of the invention, there are provided processes for utilizing polypeptides or polynucleotides of the invention, for in vitro purposes related to scientific research, synthesis of DNA, and manufacture of vectors.

In another embodiment of the invention there is provided a computer readable medium having stored thereon a member selected from the group consisting of: (a) a polynucleotide comprising the sequence of SEQ ID NO. 1 or 3; (b) a polypeptide comprising the sequence of SEQ ID NO. 2 or 4; (c) a data set of polynucleotide sequences wherein at least one of said sequences comprises the sequence of SEQ ID NO. 1 or 3; (d) a data set of polypeptide sequences wherein at least one of said sequences comprises the sequence of SEQ ID NO. 2 or 4; (e) a data set representing a polynucleotide sequence comprising the sequence of SEQ ID NO. 1 or 3; and (f) a data set representing a polynucleotide sequence encoding a polypeptide sequence comprising the sequence of SEQ ID NO. 2 or 4.

A further embodiment of the invention provides a computer based method for performing homology identification, said method comprising the steps of providing a polynucleotide sequence comprising the sequence of SEQ ID NO. 1 or 3 in a computer readable medium; and comparing said polynucleotide sequence to at least one polynucleotide or polypeptide sequence to identify homology.

A further embodiment of the invention provides a computer based method for performing homology identification, said method comprising the steps of: providing a polypeptide sequence comprising the sequence of SEQ ID NO. 2 or 4 in a computer readable medium; and comparing said polypeptide sequence to at least one polynucleotide or polypeptide sequence to identify homology.

A further embodiment of the invention provides a computer based method for polynucleotide assembly, said method comprising the steps of: (a) providing a first polynucleotide sequence comprising the sequence of SEQ ID NO. 1 or 3 in a computer readable medium; and (b) screening for at least one overlapping region between said first polynucleotide sequence and a second polynucleotide sequence.

A further embodiment of the invention provides a computer based method for performing homology identification, said method comprising the steps of: (a) providing a polynucleotide sequence comprising the sequence of SEQ ID NO. 1 or 3 in a computer readable medium; and (b) comparing said polynucleotide sequence to at least one polynucleotide or polypeptide sequence to identify homology.

A further embodiment of the invention provides a computer based method for performing homology identification, said method comprising the steps of: (a) providing a polypeptide sequence comprising the sequence of SEQ ID NO. 2 or 4 in a computer readable medium; and (b) comparing said polypeptide sequence to at least one polynucleotide or polypeptide sequence to identify homology.

A further embodiment of the invention provides a computer based method for polynucleotide assembly, said method comprising the steps of: (a) providing a first polynucleotide sequence comprising the sequence of SEQ ID NO. 1 or 3 in a computer readable medium; and (b) screening for at least one overlapping region between said first polynucleotide sequence and a second polynucleotide sequence.

Various changes and modifications within the spirit and scope of the disclosed invention will become readily apparent to those skilled in the art from reading the following descriptions and from reading the other parts of the present disclosure.

These and other aspects, features, and advantages of the present invention should be apparent to those skilled in the art from the following drawings and detailed description.

### BRIEF DESCRIPTION OF THE DRAWINGS

The invention will now be described in relation to the drawings in which:

FIG. 1 are blots showing Silver stained SDS-PAGE gel of whole cell LPS extracts of S. typhimurium strains showing complementation of WaaF deficient mutations by the H. pylori HP1191 gene.- Lane 1-S. typhimurium strain 3370+pUC19; Lane 2-S. typhimurium strain 3770+p1191; Lane 3-S. typhimurium strain 3789+pUC19; Lane 4-S. typhimurium strain 3789+p1191.

FIG. 2 are blots showing Silver stained SDS-PAGE gel of whole cell LPS extracts of S. typhimurium strains showing complementation of WaaF and WaaC deficient mutations by the H. pylori HHP1191 gene or HP0279 genes. Lane 1-S. typhimurium strain 1355+pUC19; Lane 2-S. typhimurium strain 1377+pUC19; Lane 3-S. typhimurium strain 1355+p0279G1; Lane 4-S. typhimurium strain 1377 p0279G1; Lane 5-S. typhimurium strain 1355+p1191; Lane 6-S. typhimurium strain 1377+p1191; Lane 7-S. typhimurium strain 3770+p0279G1; Lane 8-S. typhimurium strain 3789+p0279G1.

FIG. 3 shows the molecular structure of H. pylori LPS.

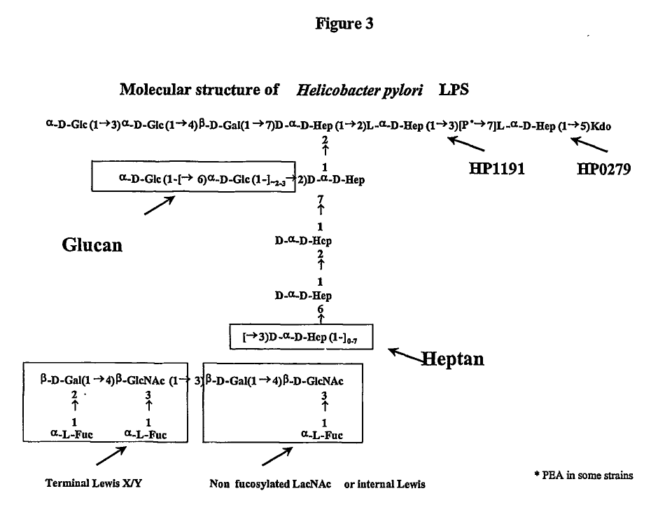

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M..J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

### Glossary

The following definitions are provided to facilitate understanding of certain terms used herein.

The term “complementary” refers to the natural binding of polynucleotides under permissive salt and temperature conditions by base-pairing. For example, the sequence “A—G—T” binds to the complementary sequence “T—C—A”. Complementarity between two single-stranded molecules may be “partial”, in which only some of the nucleic acids bind, or it may be complete when total complementarity exists between the single stranded molecules.

The term “consisting essentially of” or “consisting of” a polynucleotide sequence refers to the disclosed polynucleotide sequence, and also encompasses polynucleotide sequences which are identical except for a base change or substitution therein. As known to those skilled in the art, a limited number of base changes or substitutions may be made in a short oligonucleotide sequence resulting in a sequence maintaining substantial function (ranging from approximately 50% to greater than 100% of the activity) of the original unmodified sequence.

“Disease(s)” means a condition or disease caused by or related to infection by a bacteria that comprises a polypeptide or polynucleotide of the invention.

“Host cell” is a cell which has been transformed or transfected, or is capable of being transformed or transfected by an exogenous polynucleotide sequence.

“Identity,” as known in the art, is a relationship between two or more polypeptide sequences or two or more polynucleotide sequences, as determined by comparing the sequences. “Identity” also refers to the degree of sequence relatedness between polypeptide or polynucleotide sequences as determined by the match between strings of such sequences. “Identity” may be calculated by conventional methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48. 1073 (1988). Methods to determine identity are designed to give the highest match between the sequences tested. Methods to determine identity are codified in publicly available computer programs. Examples of computer program methods to determine identity between two sequences include, but are not limited to, the GCG program package (Devereux, J., et al., Nucleic Acids Research 12(1). 387 (1984)), BLASTP, BLASTN, and FASTA (Atschul, S. F. et al., J. Molec. Biol 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403410 (1990). The Smith Waterman algorithm known in the art may also be used to determine identity.

Parameters for comparison of polypeptide sequences include the following: (1) Algorithm: Needleman and Wunsch, J. Mol Biol. 48: 443-453 (1970); (2) Comparison matrix: BLOSSUM62 from Hentikoff and Hentikoff, Proc. Natl. Acad. Sci. USA. 89:10915-10919 (1992); (3) Gap Penalty: 12; and (4) Gap Length Penalty: 4. A useful publicly available program with these parameters is the “gap” program from Genetics Computer Group, Madison Wis. The above-mentioned comparison parameters are the default parameters for peptide comparisons (along with no penalty for end gaps).

Parameters for comparison of polynucleotide sequences include the following: (1) Algorithm: Needleman and Wunsch, J. Mol Biol. 48: 443-453 (1970); (2) Comparison matrix: matches=+10, mismatch=0; (3) Gap Penalty: 50; and (4) Gap Length Penalty: 3. The “gap” program from Genetics Computer Group, Madison, Wis. is a publicly available program with these default parameters for nucleic acid comparisons.

A preferred meaning for “identity” for polynucleotides and polypeptides is as follows:

(1) Polynucleotide embodiments may include an isolated polynucleotide comprising a polynucleotide sequence having at least 50, 60, 70, 80, 85, 90, 95, 97 or 100% identity to the sequence of SEQ ID NO: 1 or 3, where the polynucleotide sequence may be identical to the sequence of SEQ ID NO: 1 or 3 or may include up to a certain integer number of nucleotide alterations as compared to the sequence of SEQ ID NO: 1 or 3. The alterations may be selected from the group consisting of at least one nucleotide deletion, substitution, including transition and transversion, or insertion. The alterations may occur at the 5′ or 3′ terminal positions of the sequence of SEQ ID NO: 1 or 3 or anywhere between those terminal positions, interspersed either individually among the nucleotides in the sequence of SEQ ID NO: 1 or 3 or in one or more contiguous groups within this sequence. The number of nucleotide alterations can be determined by multiplying the total number of nucleotides in SEQ ID NO: 1 or 3 by the integer defining the percent identity divided by 100 and then subtracting that product from the total number of nucleotides in SEQ ID NO: 1 or 3.

(2) Polypeptide embodiments may include an isolated polypeptide comprising a polypeptide having at least a 50, 60, 70, 80, 85, 90, 95, 97 or 100% identity to a polypeptide sequence of SEQ ID NO: 2 or 4 where the polypeptide sequence may be identical to the sequence of SEQ ID NO: 2 or 4 or may include up to a certain integer number of amino acid alterations as compared to the sequence. The alterations may be selected from the group consisting of at least one amino acid deletion, substitution, including conservative and non-conservative substitution, or insertion, and where the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations is determined by multiplying the total number of amino acids in SEQ ID NO: 2 or 4 by the integer defining the percent identity divided by 100 and then subtracting that product from said total number of amino acids in SEQ ID NO: 2 or 4.

The term “isolated” refers to a polynucleotide or polypeptide changed and/or removed from its natural environment, purified or separated, or substantially free of cellular material or culture medium when produced by recombinant DNA techniques, or chemical reactants, or other chemicals when chemically synthesized. A polynucleotide or polypeptide that is introduced into an organism by transformation, genetic manipulation, or any other recombinant method is “isolated” even if it is still present in an organism, which may be living or non-living. Preferably, an isolated polynucleotide or polypeptide is at least 60% free, more preferably at least 75% free, and most preferably at least 90% free from other components with which they are naturally associated.

“Polynucleotide(s)” generally refers to any polyribonucleotide or polydeoxribonucleotide, which may be unmodified RNA or DNA or modified RNA or DNA, including mRNAs, DNAs, cDNAs and genomic DNA. “Polynucleotide(s)” include, without limitation, single- and double-stranded DNA, DNA that is a mixture of single- and double-stranded regions or single-, double- and triple-stranded regions, single- and double stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also includes triple-stranded regions comprising RNA or DNA or both RNA and DNA. The strands in such triple-stranded regions may be from the same molecule or from different molecules. The regions may include all or one or more of the molecules, but typically involve only a region of some of the molecules. One of the molecules of a triple-helical region often is an oligonucleotide. As used herein, the term “polynucleotide(s)” also includes DNAs or RNAs as described herein that contain one or more modified bases. Thus, DNAs or RNAs with backbones modified for stability or for other reasons are within the meaning of the term “polynucleotide(s)”. “Polynucleotide(s)” also includes DNAs or RNAs comprising unusual bases, such as inosine, or modified bases, such as tritylated bases, to name just two examples. A great variety of modifications have been made to DNA and RNA that serve many useful purposes known to those of skill in the art and the term “polynucleotide(s)” embraces such chemically, enymatically or metabolically modified forms of polynucleotides, as well as the chemical forms of DNA and RNA characteristic of viruses and cells, including, for example, simple and complex cells. “Polynucleotide(s)” also includes short polynucleotides often referred to as oligonucleotide(s). The term “polynucleotides” and in particular DNA or RNA, refers only to the primary and secondary structure and it does not limit it to any particular tertiary forms.

The term “polynucleotide encoding a polypeptide” encompasses polynucleotides that include a sequence encoding a polypeptide of the invention, particularly a bacteria polypeptide and more particularly a polypeptide of Helicobacter pylori having an amino acid sequence set out in SEQ ID NO: 2 or 4. The term also contemplates polynucleotides that include a single continuous region or discontinuous regions encoding the polypeptide (e.g. interrupted by integrated phage or an insertion sequence or editing) together with additional regions, that also may contain coding and/or non-coding sequences.

“Polypeptide(s)” refers to any peptide or protein comprising two or more amino acids joined to each other by peptide bonds or modified peptide bonds. The term includes both short chains, commonly referred to as peptides, oligopeptides and oligomers and to longer chains generally referred to as proteins. Polypeptides may contain amino acids other than the 20 gene encoded amino acids. “Polypeptide(s)” as used herein includes those modified either by natural processes, such as processing and other post-translational modifications, but also by chemical modification techniques. Such modifications are well described in basic texts and research literature, and they are well known to those of skill in the art. The same type of modification may be present in the same or varying degree at several sites in a given polypeptide, and a given polypeptide may contain many types of modifications. Modifications may occur anywhere in a polypeptide, including the peptide backbone, the amino acid side-chains, and the amino or carboxyl termini. Examples of modifications include, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. (See, for example, PROTEINS—STRUCTURE AND MOLECULAR PROPERTIES, 2^{nd} Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). “Polypeptides” may be branched or cyclic, with or without branching. These polypeptides may result from post-translational natural processes and may be made by entirely synthetic methods.

“Variant(s)” as used herein refers to a polynucleotide or polypeptide that differs from a reference polynucleotide or polypeptide respectively, but retains essential properties. A typical variant of a polynucleotide differs in nucleotide sequence from another polynucleotide. Changes in the nucleotide sequence of the variant may or may not alter the amino acid sequence of an encoded polypeptide. Nucleotide changes may result in amino acid substitutions, additions, deletions, fusions and truncations in the polypeptide encoded by the reference sequence. A typical variant of a polypeptide differs in amino acid sequence from another, reference polypeptide. Differences are generally limited so that the sequences of the reference polypeptide and the variant are very similar overall and, in many regions, identical. A variant may differ in amino acid sequence by one or more substitutions, additions, deletions in any combination. A substituted or inserted amino acid residue may or may not be one encoded by the genetic code. A variant of a polynucleotide or polypeptide may be a naturally occurring variant such as an allelic variant, or it may be a variant that is not known to occur naturally. Mutagenesis techniques, direct synthesis, and other recombinant methods known to skilled artisans may be used to produce non-naturally occurring variants of polynucleotides and polypeptides.

A “ligand” refers to a compound or entity that associates with a polypeptide of the invention or part thereof, including acceptor molecules or analogues or parts thereof, and donor molecules or analogues or parts thereof.

A “donor molecule” refers to a molecule capable of donating a sugar to an acceptor molecule, via the action of a HepT polypeptide. The donor molecule may be di- or poly-saccharides, sugar 1-phosphates, or, most commonly, nucleotide diphosphosugars (ADP-sugars), or nucleotide phosphosugars. In an embodiment, the donor molecule for an ADP heptose-LPS-heptosyltransferase is ADP-mannoheptose.

An acceptor molecule is capable of accepting a sugar from a donor molecule, via the action of a HepT polypeptide. It may, for example, comprise a terminal sugar residue for transfer purposes. The acceptor molecule or aglycone can be, for example, a lipid, a protein, a heterocyclic compound, an antibiotic, a peptide, an amino acid, an aromatic or aliphatic alcohol or thiol or another carbohydrate residue. In a preferred embodiment, the acceptor molecule is or comprises a terminal L-α-D-heptose (for HP1191) or KDO (for HP0279).

An analogue of a donor or acceptor molecule is one which mimics the donor or acceptor molecule binding to a HepT polypeptide but which is incapable (or has a significantly reduced capacity) to take part in the transfer reaction.

Polynucleotides

As hereinbefore mentioned, the invention provides isolated polynucleotides, (including full length hepT genes) that encode HepT polypeptides, or fragments, variants, homologs thereof, and polynucleotides having substantial identity thereto, and variants thereof. Preferably, the polynucleotides encode polypeptides that retain substantially the same biological function or activity of a mature HepT.

In an embodiment of the invention an isolated polynucleotide is contemplated which comprises:

(i) a polynucleotide encoding a polypeptide having substantial sequence identity, preferably at least 50%, more preferably at least 70% sequence identity, with an amino acid sequence of SEQ. ID. NO. 2 or 4;

(ii) polynucleotides complementary to (i);

(iii) polynucleotides differing from any of the polynucleotides of (i) or (ii) in codon sequences due to the degeneracy of the genetic code;

(iv) a polynucleotide comprising at least 10, 15, or 18, preferably at least 20 nucleotides and capable of hybridizing under stringent conditions to a polynucleotide of SEQ. ID. NO. 1 or 3 or to a degenerate form thereof;

(v) a polynucleotide encoding an allelic or species variation of a polypeptide comprising an amino acid sequence of SEQ. ID. NO. 2 or 4; or

(vi) a fragment, or allelic or species variation of (i), (ii) or (iii)

In a specific embodiment, the isolated polynucleotide comprises:

(i) a polynucleotide having substantial sequence identity, preferably at least 50%, more preferably at least 70% sequence identity with a sequence of SEQ. ID. NO. 1 or 3;

(ii) polynucleotides complementary to (i), preferably complementary to a full sequence of SEQ. ID. NO. 1 or 3;

(iii) polynucleotides differing from any of the nucleic acids of (i) to (ii) in codon sequences due to the degeneracy of the genetic code; or

(iv) a fragment, or allelic or species variation of (i), (ii) or (iii).

In a preferred embodiment the isolated polynucleotide encodes a polypeptide comprising or consisting essentially of an amino acid sequence of SEQ. ID. NO. 2 or 4, or comprises or consists essentially of a polynucleotide of SEQ. ID. NO. 1 or 3 wherein T can also be U. The DNA sequence set out in SEQ ID NO: 1 or 3 contains an open reading frame encoding a polypeptide comprising the amino acid residues set forth in SEQ ID NO: 2 or 4, respectively, with a deduced molecular weight that can be calculated using amino acid residue molecular weight values well known in the art.

Preferably, a polynucleotide of the present invention has substantial sequence identity using the preferred computer programs cited herein, for example at least 50%, 60%, 70%, 75%, 80%, 90%, more preferably at least 95%, 96%, 97%, 98%, or 99% sequence identity to a sequence of SEQ. ID. NO. 1 or 3.

Isolated nucleic acid molecules encoding a polypeptide of the invention and having a sequence which differs from a polynucleotide of SEQ. ID. NO. 1 or 3 due to degeneracy in the genetic code are also within the scope of the invention. As one example, DNA sequence variations within a hepTgene may result in silent mutations which do not affect the amino acid sequence. Variations in one or more nucleotides may exist among organisms within a genus due to natural allelic variation. Any and all such nucleic acid variations are within the scope of the invention. DNA sequence variations may also occur which lead to changes in the amino acid sequence of a polypeptide of the invention. These amino acid variations are also within the scope of the present invention. In addition, species variations i.e. variations in nucleotide sequence naturally occurring among different species, are within the scope of the invention.

The invention contemplates the coding sequence for a mature polypeptide or a fragment thereof, by itself as well as the coding sequence for a mature polypeptide or a fragment in reading frame with other coding sequences, including those encoding a leader or secretory sequence, a pre-, or pro- or prepro- protein sequence. A polynucleotide of the invention may also contain non-coding sequences, including, but not limited to non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. The additional sequences may be a marker sequence that facilitates purification of the fused polypeptide, the sequences may play a role in processing of a polypeptide from precursor to a mature form, may allow protein transport, may lengthen or shorten protein half-life or may facilitate manipulation of a protein for assay or production. Additional sequences may be at the amino or carboxyl-terminal end or interior to the mature polypeptide.

Polynucleotides of the invention also include, but are not limited to, polynucleotides comprising a structural gene and its naturally associated sequences that control gene expression.

Also included in the invention are polynucleotides of the formula:

X—(R₁)ₘ—(Z)—(R₂)ₙ—Y

wherein, at the 5′ end of the molecule, X is hydrogen or a metal or together with Y defines a covalent bond, and at the 3′ end of the molecule, Y is hydrogen or a metal or together with X defmes a covalent bond, each occurrence of R₁ and R₂ is independently any nucleic acid residue, m is an integer between 1 and 3000 or zero, preferably between 1 and 1000, n is an integer between 1 and 3000 or zero, preferably between 1 and 1000, and Z is a polynucleotide sequence of the invention, particularly a sequence selected from SEQ ID NO: 1 or 3. Any stretch of nucleotide residues denoted by either R group, where m and/or n is greater than 1, may be either a heteropolymer or a homopolymer, preferably a heteropolymer. In an embodiment, X and Y together define a covalent bond and the polynucleotide of the above formula is a closed circular polynucleotide, which can be a double-stranded polynucleotide wherein the formula shows a first strand to which the second strand is complementary.

Fragments of a polynucleotide of the invention, include fragments that are a stretch of at least about 10, 15, 18, 20, 40, 50, 100, or 150 nucleotides, more typically at least 50 to 100 nucleotides but less than 2 kb. It will further be appreciated that variant forms of the polynucleotides of the invention which arise by alternative splicing of an mRNA corresponding to a cDNA of the invention are encompassed by the invention. Polynucleotides that encode for variants of polypeptides of the invention are particularly contemplated that have an amino acid sequence of SEQ ID NO: 2 or 4, in which several, a few, 5 to 10, 1 to 5, 1 to 3, 2, 1, or no amino acid residues are substituted. Preferred among these variants are silent substitutions, additions, and deletions that do not alter the properties and activities of the polypeptide.

Another aspect of the invention provides a polynucleotide which hybridizes under selective conditions, e.g. high stringency conditions, to a polynucleotide which comprises a sequence which encodes a polypeptide of the invention. Preferably the sequence encodes an amino acid sequence of SEQ. ID. NO. 2 or 4 or part thereof and comprises at least 18 nucleotides. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N.Y. (1989), 6.3.1-6.3.6. For example, hybridization may occur at 30° C. in 750 mM NaCl, 75 mM trisodium citrate, and 1% SDS, preferably 37° C. in 500 mM NaCl, 500 mM trisodium citrate, 1% SDS, 35% formamide, and 100 μg/ml denatured salmon sperm DNA (ssDNA), and more preferably 42° C. in 250 mM NaCl, 25 mM trisodium citrate, 1% SDS, 50% formamide, and 200 μg/ml ssDNA. Useful variations on these conditions will be readily apparent to those skilled in the art.

The stringency may be selected based on the conditions used in the wash step. Wash step stringency conditions may be defined by salt concentration and by temperature. Generally, wash stringency can be increased by decreasing salt concentration or by increasing temperature. By way of example, a stringent salt concentration for the wash step is preferably less than about 30 mM NaCl and 3 mM trisodium citrate, and more preferably less than about 15 mM NaCl and 1.5 mM trisodium citrate. Stringent temperature conditions will generally include temperatures of a least about 25° C. , more preferably at least about 68° C. . In a preferred embodiment, the wash steps will be carried out at 42° C. in 15 mM NaCl, 1.5 mM trisodium citrate, and 0.1% SDS. In a more preferred embodiment the wash steps are carried out at 68° C. in 15 mM NaCl, 1.5 mM trisodium citrate, and 0.1% SDS. Variations on these conditions will be readily apparent to those skilled in the art.

The polynucleotides of the inventions are preferably derived from Helicobacter pylori, however, they may be obtained from organisms of the same taxonomic genus. They may also be obtained from organisms of the same taxonomic family or order.

An isolated polynucleotide molecule of the invention which comprises DNA can be isolated by preparing a labeled nucleic acid probe based on all or part of a nucleic acid sequence of SEQ. ID. NO. 1 or 3. The labeled nucleic acid probe is used to screen an appropriate DNA library (e.g. a cDNA or genomic DNA library). For example, a cDNA library can be used to isolate a cDNA encoding a polypeptide of the invention by screening the library with the labeled probe using standard techniques. Alternatively, a genomic DNA library can be similarly screened to isolate a genomic clone encompassing a hepT gene. Polynucleotides isolated by screening of a cDNA or genomic DNA library can be sequenced by standard techniques.

An isolated polynucleotide of the invention which is DNA can also be isolated by selectively amplifying a polynucleotide of the invention. “Amplifying” or “amplification ” refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.). In particular, it is possible to design synthetic oligonucleotide primers from a nucleotide sequence of SEQ. ID. NO. 1 or 3 for use in PCR. Examples of suitable primers are the sequences of SEQ ID NO. 5 through 8. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated polynucleotide of the invention which is RNA can be isolated by cloning a cDNA encoding a polypeptide of the invention into an appropriate vector which allows for transcription of the cDNA to produce an RNA molecule which encodes the polypeptide. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

A polynucleotide of the invention may be engineered using methods known in the art to alter the hepT encoding sequence for a variety of purposes including modification of the cloning, processing, and/or expression of the gene product. Procedures such as DNA shuffling by random fragmentation and PCR reassembly of gene fragments and synthetic oligonucleotides may be used to engineer the nucleic acid molecules. Mutations may be introduced by oligonucleotide-mediated site-directed mutagenesis to create for example new restriction sites, change codon preference, or produce variants.

Polynucleotides of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular polynucleotide is a hepT gene or encodes a polypeptide of the invention can be accomplished by expressing the cDNA in an appropriate host cell by standard techniques, and testing the expressed protein in the methods described herein. A cDNA encoding a polypeptide of the invention can be sequenced by standard techniques, such as dideoxynucleotide chain termination or Maxam-Gilbert chemical sequencing, to determine the nucleic acid sequence and the predicted amino acid sequence of the encoded protein.

The polynucleotides of the invention may be extended using a partial nucleotide sequence and various PCR-based methods known in the art to detect upstream sequences such as promoters and regulatory elements. For example, restriction-site PCR which uses universal and nested primers to amplify unknown sequences from genomic DNA within a cloning vector may be employed (See Sarkar, G, PCR Methods Applic. 2:318-322, 1993). Inverse PCR which uses primers that extend in divergent directions to amplify unknown sequences from a circularized template may also be used. The template in inverse PCR is derived from restriction fragments adjacent to known sequences in human and yeast artificial chromosome DNA (See e.g. Lagerstrom, M., at al, PCR Methods Applic. 1:111-119, 1991). Other methods for retrieving unknown sequences are known in the art (e.g. Parker, J. D. et al, Nucleic Acids Res. 19:305-306, 1991). In addition, PCR, nested primers, and PROMOTERFINDER libraries (Clontech, Palo Alto, Calif.) may be used to walk genomic DNA.

It is preferable when screening for full-length cDNAs to use libraries that have been size-selected to include larger cDNAs. For situations in which an oligo d(T) library does not yield a full-length cDNA, it is preferable to use random-primed libraries which often include sequences containing the 5′ regions of genes. Genomic libraries may be useful for extending the sequence into 5′ non-translated regulatory regions.

Commercially available capillary electrophoresis systems may be employed to analyse the size or confirm the sequence of PCR or sequencing products. The system may use flowable polymers for electrophoretic separation, four different nucleotide-specific, laser-stimulated fluorescent dyes, and a charge coupled device camera for detection of the emitted wavelengths. Commercially available software (e.g. GENOTYPER and SEQUENCE NAVIGATOR, Perkin-Elmer) may convert the output/light intensity to electrical signal, and the entire process from loading of samples, and computer analysis and electronic data display may be computer controlled. This procedure may be particularly useful for sequencing small DNA fragments which may be present in limited amounts in a particular sample.

In accordance with another aspect of the invention, the polynucleotides isolated using the methods described herein are mutant hepT gene alleles. For example, the mutant alleles may be isolated from organisms either known or proposed to contribute to a disease. Mutant alleles and mutant allele products may be used in therapeutic and diagnostic methods described herein. For example, a cDNA of a mutant hepT gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an organism suspected of or known to carry a mutant allele, or a cDNA library can be constructed using RNA from organisms known to express the mutant allele. A polynucleotide encoding a normal hepT gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from organisms known or suspected to express a mutant hepT allele. Gene products from putatively mutant organisms may be expressed and screened, for example using antibodies specific for a polypeptide as described herein. Library clones identified using the antibodies can be purified and subjected to sequence analysis.

Antisense molecules and ribozymes are contemplated within the scope of the invention. “Antisense” refers to any composition containing nucleotide sequences which are complementary to a specific DNA or RNA sequence. Ribozymes are enzymatic RNA molecules that can be used to catalyze the specific cleavage of RNA. Antisense molecules and ribozymes may be prepared by any method known in the art for the synthesis of polynucleotides. These include techniques for chemically synthesizing oligonucleotides such as solid phase phosphoramidite chemical synthesis. Alternatively, RNA molecules may be generated by in vitro and in vivo transcription of DNA sequences encoding a polypeptide of the invention. Such DNA sequences may be incorporated into a wide variety of vectors with suitable RNA polymerase promoters such as T7 or SP6. Alternatively, these cDNA constructs that synthesize antisense RNA constitutively or inducibly can be introduced into organisms. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Polypeptides

A polypeptide of the invention includes a polypeptide of SEQ. ID. NO: 2 or 4, particularly those which have the biological activity of a HepT polypeptide. In addition to polypeptides comprising an amino acid sequence of SEQ.ID. NO. 2 or 4 the polypeptides of the present invention include truncations or fragments, and variants, and homologs.

Truncated polypeptides may comprise peptides of between 3 and 70 amino acid residues, ranging in size from a tripeptide to a 50 mer polypeptide, preferably 30 to 50 amino acids. In one aspect of the invention, fragments of a polypeptide of the invention are provided having an amino acid sequence of at least five consecutive amino acids of SEQ. ID. NO. 2 or 4 where no amino acid sequence of five or more, six or more, seven or more, or eight or more, consecutive amino acids present in the fragment is present in a polypeptide other than a HepT of the invention. In an embodiment of the invention the fragment is a stretch of amino acid residues of at least 12 to 20 contiguous amino acids from particular sequences such as the sequences of SEQ.ID. NO. 2 or 4. The fragments may be immunogenic and preferably are not immunoreactive with antibodies that are immunoreactive to polypeptides other than a HepT of the invention. Particularly preferred are fragments that are antigenic or immunogenic in an animal, especially in a human.

A fragment may be characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding regions, and high antigenic index regions.

In a preferred embodiment, the invention provides biologically active fragments which are those fragments that mediate activities of a HepT polypeptide, including those with a similar activity or an improved activity, or with a decreased undesirable activity. Particularly preferred are fragments comprising domains of enzymes that confer a function essential for viability of Helicobacter species or the ability to initiate, maintain, or cause disease in an individual, particularly a human.

Truncated polypeptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated polypeptides may have a carboxyl group, an amido group, a Tbutyloxycarbonyl group, or a macromolecule including but not limited to lipid-fatty acid conjugates, polyethylene glycol, or carbohydrates at the carboxy terminal end.

A truncated polypeptide or fragment may be “free-standing,” or comprised within a larger polypeptide of which they form a part or region, most preferably as a single continuous region, of a single larger polypeptide.

The polypeptides of the invention may also include variants of a HepT of the invention, and/or truncations thereof as described herein, which may include, but are not limited to a polypeptide of the invention containing one or more amino acid substitutions, insertions, and/or deletions. Amino acid substitutions may be of a conserved or non-conserved nature. Conserved amino acid substitutions involve replacing one or more amino acids of a HepT amino acid sequence with amino acids of similar charge, size, and/or hydrophobicity characteristics. When only conserved substitutions are made the resulting analog is preferably functionally equivalent to a HepT of the invention. Non-conserved substitutions involve replacing one or more amino acids of the HepT amino acid sequence with one or more amino acids which possess dissimilar charge, size, and/or hydrophobicity characteristics.

One or more amino acid insertions may be introduced into a polypeptide of the invention. Amino acid insertions may consist of single amino acid residues or sequential amino acids ranging from 2 to 15 amino acids in length.

Deletions may consist of the removal of one or more amino acids, or discrete portions from the HepT amino acid sequence. The deleted amino acids may or may not be contiguous. The lower limit length of the resulting analog with a deletion mutation is about 10 amino acids, preferably 50 amino acids.

Allelic variants of a HepT at the protein level differ from one another by only one, or at most, a few amino acid substitutions. A species variation of a HepT polypeptide is a variation which is naturally occurring among different species of an organism.

The polypeptides of the invention include homologs of a HepT and/or truncations thereof as described herein. Such HepT homologs include proteins whose amino acid sequences are comprised of the amino acid sequences of HepT regions from other species that hybridize under selective hybridization conditions (see discussion of selective and in particular stringent hybridization conditions herein) with a probe used to obtain a polypeptide. These homologs will generally have the same regions which are characteristic of a HepT polypeptide. It is anticipated that a protein comprising an amino acid sequence which has at least 50%, 60%, 70%, 75%, 80%, 85%, or 90% identity, more preferably 95%, 96%, 97%, 98%, or 99% identity with an amino acid sequence of SEQ. ID. NO. 2 or 4 will be a homolog of a polypeptide of the invention. A percent amino acid sequence homology or identity is calculated using the methods described herein, preferably the computer programs described herein.

The invention also contemplates isoforms of polypeptides of the invention. An isoform contains the same number and kinds of amino acids as a polypeptide of the invention, but the isoform has a different molecular structure. The isoforms contemplated by the present invention preferably have the same properties as a polypeptide of the invention as described herein.

The present invention also provides a polypeptide of the invention conjugated with a selected protein, or a marker (see below), or other glycosyltransferase, to produce fusion proteins or chimeric proteins.

Also included in the invention are polypeptides of the formula:

X—(R₁)ₘ—(Z)—(R₂)ₙ—Y

wherein, at the amino terminus, X is hydrogen or a metal, and at the carboxy terminus Y is hydrogen or a metal, or together Y and X define a covalent bond, each occurrence of R₁ and R₂ is independently any amino acid residue, m is an integer between 1 and 1000 or zero, preferably between 1 and 1000, n is an integer between 1 and 3000 or zero, preferably between 1 and 1000, and Z is a polypeptide of the invention, particularly a sequence selected from SEQ ID NO: 2 or 4. Any stretch of amino acid residues denoted by either R group, where m and/or n is greater than 1, may be either a heteropolymer or a homopolymer, preferably a heteropolymer. Where, in a preferred embodiment, X and Y together define a covalent bond, the polypeptide of the above formula is a closed, circular polypeptide.

A polypeptide of the invention may be prepared using recombinant DNA methods. Accordingly, polynucleotides of the present invention having a sequence which encodes a polypeptide of the invention may be incorporated in a known manner into an appropriate expression vector which ensures good expression of the polypeptide. Possible expression vectors include but are not limited to chromosomal, episomal and virus-derived vectors, so long as the vector is compatible with the host cell used. Representative examples of vectors are vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids

The invention therefore contemplates a recombinant expression vector comprising a polynucleotide of the invention, and the necessary regulatory sequences for the transcription and translation of the inserted sequence. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. The necessary regulatory sequences may be supplied by the native polypeptide and/or its flanking regions.

The invention further provides a recombinant expression vector comprising a polynucleotide of the invention cloned into the expression vector in an antisense orientation. That is, the DNA molecule is linked to a regulatory sequence in a manner which allows for expression, by transcription of the DNA molecule, of an RNA molecule which is antisense to a polynucleotide sequence of SEQ. ID. NO. 1 or 3. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418, dhfr, npt, als, pat and hygromycin which confer resistance to certain drugs, β-galactosidase, chloramphenicol acetyltransferase, firefly luciferase, trpB, hisD, herpes simplex virus thymidine kinase, adenine phosphoribosyl transferase, or an immunoglobulin or portion thereof such as the Fc portion of an imnuunoglobulin preferably IgG. Visible markers such as anthocyanins, beta-glucuronidase and its substrate GUS, and luciferase and its substrate luciferin, can be used to identify transformants, and also to quantify the amount of transient or stable protein expression attributable to a specific vector system (Rhodes, C. et al. (1995)Mol. Biol. 55:121-131). The markers can be introduced on a separate vector from the nucleic acid of interest.

The recombinant expression vectors may also contain genes that encode a fusion moiety which provides increased expression of the recombinant polypeptide; increased solubility of the recombinant polypeptide; and aid in the purification of the target recombinant polypeptide by acting as a ligand in affinity purification. For example, a proteolytic cleavage site may be added to the target recombinant polypeptide to allow separation of the recombinant polypeptide from the fusion moiety subsequent to purification of the fusion protein. Typical fusion expression vectors include pGEX (Amrad Corp., Melbourne, Australia), pMAL (New England Biolabs, Beverly, Mass.) and pRITS (Pharmacia, Piscataway, N.J.) which fuse glutathione S-transferase (GST), maltose E binding protein, or protein A, respectively, to the recombinant protein.

The vectors may be introduced into host cells to produce a transformed or transfected host cell. The terms “transfected ” and “transfection” encompass the introduction of nucleic acid (e.g. a vector) into a cell by one of many standard techniques. A cell is “transformed” by a nucleic acid when the transfected nucleic acid effects a phenotypic change. Prokaryotic cells can be transfected or transformed with nucleic acid by, for example, electroporation or calcium-chloride mediated transformation. Nucleic acid can be introduced into marmmalian cells via conventional techniques such as calcium phosphate or calcium chloride co-precipitation, DEAE-dextranmediated transfection, lipofectin, electroporation or microinjection. Suitable methods for transforming and transfecting host cells can be found in Sambrook et al. (Molecular Cloning: A Laboratory Manual, 2^{nd} Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

Examples of appropriate host cells include bacterial cells, such as Streptococci, Staphylococci, Enterococci, E. coli Helicobacter, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A host cell may also be chosen which modulates the expression of an inserted nucleic acid sequence, or modifies (e.g. glycosylation) and processes (e.g. cleaves) the polypeptide in a desired fashion. Host systems or cell lines may be selected which have specific and characteristic mechanisms for post-translational processing and modification of proteins. For long-term high-yield stable expression of the polypeptide, cell lines and host systems which stably express the gene product may be engineered.

Host cells and in particular cell lines produced using the methods described herein may be particularly useful in screening and evaluating compounds that modulate the activity of a polypeptide of the invention.

Polypeptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). Protein synthesis may be performed using manual procedures or by automation. Automated synthesis may be carried out, for example, using an Applied Biosystems 431A peptide synthesizer (Perkin Elmer). Various fragments of the polypeptides of the invention may be chemically synthesized separately and combined using chemical methods to produce the full length molecule.

N-terminal or C-terminal fusion polypeptides or chimeric polypeptides comprising a polypeptide of the invention conjugated with other molecules, (e.g. markers) may be prepared by fusing, through recombinant techniques, the N-terminal or C-terminal of a polypeptide of the invention, and the sequence of a selected molecule with a desired biological function (e.g.marker protein). The resultant fusion proteins contain a polypeptide of the invention fused to the selected molecule as described herein. Examples of molecules which may be used to prepare fusion proteins include imnmunoglobulins, glutathione-S-transferase (GST), protein A, hemagglutinin (HA), and truncated myc.

Antibodies

Polypeptides of the invention, or cells expressing them can be used as an immunogen to produce antibodies immunospecific for such polypeptides. “Antibodies” as used herein includes monoclonal and polyclonal antibodies, chimeric, single chain, simianized antibodies and humanized antibodies, as well as Fab fragments, including the products of an Fab immunoglobulin expression library.

In an embodiment of the invention, oligopeptides, peptides, or fragments used to induce antibodies to a polypeptide of the invention have an amino acid sequence consisting of at least 5 amino acids and more preferably at least 10 amino acids. The oligopeptides, etc. can be identical to a portion of the amino acid sequence of the natural protein, and they may contain the entire amino acid sequence of a small, naturally occurring molecule. Antibodies having specificity for a polypeptide of the invention may also be raised from fusion proteins created by expressing fusion proteins in bacteria as described herein.

Antibodies including monoclonal and polyclonal antibodies, fragments and chimeras, etc. may be prepared using methods known to those skilled in the art. Antibodies against polypeptides of the invention can be obtained by administering the polypeptides or epitope-bearing fragments, analogues or cells to an animal, preferably a nonhuman, using routine protocols. Monoclonal antibodies may be obtained by any technique known in the art that provides antibodies produced by continuous cell line cultures. (See for example, Kohler, G. and Milstein, C., Nature 256. 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Single chain antibodies to polypeptides of this invention can be prepared using methods known in the art (e.g. U.S. Pat. No. 4,946,778). Transgenic mice, or other organisms such as other mammals, may be used to express humanized antibodies.

Phage display technology may also be utilized to select antibody genes with binding activities towards a polypeptide of the invention either from repertoires of PCR amplified v-genes of lymphocytes from humans screened for possessing anti-HepT or from naive libraries (McCafferty, J. et al., (1990), Nature 348, 552-554; Marks, J. et al., (1992) Biotechnology 10, 779-783). Chain shuffling can also be used to improve the affinity of these antibodies (Clackson, T. et al., (1991) Nature 352, 624-628).

Applications

The polynucleotides, polypeptides, and antibodies of the invention may be used in the prognostic and diagnostic evaluation of disease. (See below). Methods for detecting polynucleotides and polypeptides of the invention, can be used to monitor disease in eukaryotes particularly mammals, and especially humans, particularly those infected or suspected to be infected with an organism comprising a hepT gene or polypeptide of the invention, by detecting and localizing the polynucleotides and polypeptides. The applications of the present invention also include methods for the identification of agents (e.g. compounds) which modulate the biological activity of a polypeptide of the invention (See below). The compounds, antibodies, etc. may be used for the treatment of disease. (See below).

Diagnostic and Prognostic Methods

A variety of methods can be employed for the diagnostic and prognostic evaluation of disease. Such methods may, for example, utilize polynucleotides of the invention, and fragments thereof, and antibodies of the invention. In particular, the polynucleotides and antibodies may be used, for example, for: (1) the detection of the presence of hepT gene mutations, or the detection of either over- or under-expression of HepT mRNA relative to a non-disorder state; and (2) the detection of either an over- or an under-abundance of a polypeptide of the invention relative to a non-disorder state or the presence of a modified (e.g., less than full length) polypeptide of the invention.

The methods described herein may be performed by utilizing pre-packaged diagnostic kits comprising at least one specific polynucleotide or antibody described herein, which may be conveniently used, e.g., in clinical settings, to screen and diagnose individuals and to screen and identify or monitor disease in individuals.

Nucleic acid-based detection techniques and peptide detection techniques are described below. The samples that may be analyzed using the methods of the invention include those which are known or suspected to contain a polynucleotide or polypeptide of the invention. The methods may be performed on biological samples including but not limited to cells, lysates of cells which have been incubated in cell culture, genomic DNA (in solutions or bound to a solid support such as for Southern analysis), RNA (in solution or bound to a solid support such as for northern analysis), cDNA (in solution or bound to a solid support), an extract from cells or a tissue (e.g. bone, muscle, cartilage, skin), and biological fluids such as serum, urine, blood, and CSF. The samples may be derived from a patient or a culture.

Methods for Detecting Polynucleotides

The invention provides a process for diagnosing disease, preferably bacterial infections, more preferably infections by Helicobacter pylori, comprising determining from a sample derived from an individual an increased level of expression of a polynucleotide of the invention. Increased or decreased expression of a polynucleotide of the inventon can be measured using any of the methods well known in the art.

A polynucleotide of the invention may be used in southern or northern analysis, dot blot, or other membrane-based technologies; in PCR technologies; or in dipstick, pin, ELISA assays or microarrays utilizing fluids or tissues from patients to detect altered expression. Such qualitative or quantitative methods are well known in the art and some methods are described below.

The polynucleotides of the invention allow those skilled in the art to construct nucleotide probes for use in the detection of polynucleotides of the invention in biological materials. Suitable probes include polynucleotides based on nucleic acid sequences encoding at least 5 sequential amino acids from regions of a polynucleotide of the invention (see SEQ. ID. No. 1 or 3), preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect hepT genes, preferably in human cells. The nucleotide probes may also be useful for example in the diagnosis or prognosis of disease, and in monitoring the progression of a disease condition, or monitoring a therapeutic treatment.

The probe may be used in hybridization techniques to detect htepTgenes. The technique generally involves contacting and incubating a sample from a patient or other cellular source with a probe of the present invention under conditions favourable for the specific annealing of the probes to complementary sequences in the nucleic acids. After incubation, the non-annealed nucleic acids are removed, and the presence of nucleic acids that have hybridized to the probe if any are detected.

The detection of polynucleotides of the invention may involve the amplification of specific gene sequences using an amplification method such as PCR, followed by the analysis of the amplified molecules using techniques known to those skilled in the art. Suitable primers can be routinely designed by one of skill in the art. (See SEQ ID NO 5 through 8). For example, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 60° C. to 72° C.

Genomic DNA may be used in hybridization or amplification assays of biological samples to detect abnormalities in cells involving HepT structure, including point mutations, insertions, and deletions. For example, direct sequencing, single stranded conformational polymorphism analyses, heteroduplex analysis, denaturing gradient gel electrophoresis, chemical mismatch cleavage, and oligonucleotide hybridization may be utilized. Mutations in the DNA sequence of a hepT gene may be used to diagnose infection and to serotype and/or classify the infectious agent.

Genotyping techniques known to one skilled in the art can be used to type polymorphisms that are in close proximity to the mutations in a hept gene. The polymorphisms may be used to identify species of organisms that are likely to cause disease.

RT-PCR may be used to detect mutations in the RNA. In particular, RT-PCR may be used in conjunction with automated detection systems such as for example GeneScan.

The primers and probes may be used in the above described methods in situ i.e directly on tissue sections (fixed and/or frozen) of patient tissue obtained from biopsies or resections.

Oligonucleotides derived from any of the polynucleotides of the invention may be used as targets in microarrays. “Microarray” refers to an array of distinct polynucleotides or oligonucleotides synthesized on a substrate, such as paper, nylon, or other type of membrane, filter, chip, glass slide, or any other suitable solid support.

The microarrays can be used to monitor the expression level of large numbers of genes simultaneously (to produce a transcript image) and to identify genetic variants, mutations, and polymorphisms. This information can be useful in determining gene function, diagnosing disease, and in developing and monitoring the activity of therapeutic agents (Heller, R et al. (1997) Proc. Natl. Acad, Sci. 94:2150-55).

The polynucleotides of the present invention are useful for chromosome identification. The sequences can be specifically targeted to, and can hybridize with a particular location on an individual microbial chromosome, particularly a Helicobacter pylori chromosome. The mapping of relevant sequences to a chromosome is an important step in correlating those sequences with genes associated with microbial pathogenicity and disease, or to precise chromosomal regions critical to the growth, survival, and/or ecological niche of an organism. The physical position of the sequence on the chromosome can be correlated with genetic map data to define a genetic relationship between the gene and another gene or phenotype by, for example, linkage analysis.

Differences in the RNA or genomic sequence between microbes of different phenotypes may also be determined. A mutation or sequence observed in some or all of the organisms of a certain phenotype but not in organisms lacking that phenotype, will likely be the causative agent for the phenotype. Thus, chromosomal regions may be identified that confer pathogenicity, growth characteristics, survival characteristics, and/or ecological niche.

The polynucleotides of the invention may be used in differential screening and differential display methods known in the art. (e.g. see Chuang et al J. Bacteriol. 175: 2026, 1993). Genes are identified which are expressed in an organism by identifying mRNA present using randomly primed RT-PCR. Pre-infection and post-infection profiles are compared to identify genes up and down regulated during infection.

Methods for Detecting Polypeptides

Antibodies specifically reactive with a polypeptide of the invention or derivatives thereof, such as enzyme conjugates or labeled derivatives, may be used to detect the polypeptides in various samples. They may be used as diagnostic or prognostic reagents and they may be used to detect abnormalities in the level of a polypeptide of the invention, or abnormalities in the structure of the polypeptides. Antibodies may also be used to screen potentially therapeutic compounds in vitro to determine their effects on a disease. In vitro irumunoassays may also be used to assess or monitor the efficacy of particular therapies. The antibodies of the invention may also be used in vitro to determine the level of HepT expression in cells genetically engineered to produce a HepT.

In an embodiment, the invention provides a diagnostic method for detecting over-expression of a polypeptide of the invention compared to normal control tissue samples. The method may be used to detect the presence of an infection.

The antibodies may be used in any known immunoassays which rely on the binding interaction between an antigenic determinant of a polypeptide of the invention, and the antibodies. Examples of such assays are radioimrunoassays, enzyme immunoassays (e.g. ELISA), immunofluorescence, immunoprecipitation, latex agglutination, hemagglutination, and histochemical tests. The antibodies may be used to detect and quantify polypeptides of the invention in a sample in order to determine its role in particular cellular events or pathological states, and to diagnose and treat such pathological states.

Antigenic polypeptides of the invention or fragments thereof may be used in immunoassays to detect antibody levels and correlations can be made with diseases such as gastroduodenal disease and with duodenal ulcer in particular. Immunoassays based on well defined recombinant antigens can be developed. Antibodies to Helicobacter pylori HepT polypeptides within biological samples such as blood or serum samples may be detected.

The antibodies of the invention may be used in immuno-histochemical analyses, for example, at the cellular and sub-subcellular level, to detect a polypeptide of the invention, to localise it to particular cells and tissues, and to specific subcellular locations, and to quantitate the level of expression.

Cytochemical techniques known in the art for localizing antigens using light and electron microscopy may be used to detect a polypeptide of the invention. Generally, an antibody of the invention may be labeled and a polypeptide may be localised in tissues and cells based upon detection of the label.

Various methods of labeling polypeptides are known in the art and may be used to label antibodies and polypeptides of the invention. Examples of detectable substances include, but are not limited to, the following: radioisotopes (e.g., ³H, ¹⁴C, ³⁵S, ¹²⁵I, ¹³¹I, fluorescent labels (e.g., FITC, rhodamine, lanthanide phosphors), luminescent labels such as luminol; enzymatic labels (e.g., horseradish peroxidase, β-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

An antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a polypeptide of the invention. By way of example, if the antibody having specificity against a polypeptide of the invention is a rabbit IgG antibody, the second antibody may be goat anti-rabbit gamma-globulin labelled with a detectable substance as described herein.

Where a radioactive label is used as a detectable substance, a polypeptide of the invention may be localized by radioautography. The results of radioautography may be quantitated by determining the density of particles in the radioautographs by various optical methods, or by counting the grains.

A polypeptide of the invention may also be detected by assaying for HepT activity as described herein. For example, a sample may be reacted with an acceptor molecule and a donor molecule under conditions where a HepT is capable of transferring the donor molecule to the acceptor molecule to produce a donor-acceptor complex.

Methods for Identifying or Evaluating Substances/Compounds

The invention provides methods for identifying substances that modulate the biological activity of a polypeptide of the invention including substances that interfere with, or enhance the activity of the polypeptide.

The substances and compounds identified using the methods of the invention include but are not limited to peptides such as soluble peptides including Ig-tailed fusion peptides, members of random peptide libraries and combinatorial chemistry-derived molecular libraries including libraries made of D- and/or L-configuration amino acids, phosphopeptides (including members of random or partially degenerate, directed phosphopeptide libraries), antibodies [e.g. polyclonal, monoclonal, humanized, antisense, oligosaccharides, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound. A substance of the invention may be a natural substrate or ligand (e.g. an acceptor or donor molecule) or a structural or functional mimetic. The substance may be a small molecule ligand in, for example, cells, cell-free preparations, chemical libraries, and natural product mixtures

Substances which modulate a polypeptide of the invention can be identified based on their ability to associate with (or bind to) a polypeptide of the invention. Therefore, the invention also provides methods for identifying substances which associate with a polypeptide of the invention. Substances identified using the methods of the invention may be isolated, cloned and sequenced using conventional techniques. A substance that associates with a polypeptide of the invention may be an agonist or antagonist of the biological or immunological activity of the polypeptide.

The term “agonist”, refers to a molecule that increases the amount of, or prolongs the duration of, or the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention (including ligands or mimetics thereof).

Substances which can associate with a polypeptide of the invention may be identified by reacting the polypeptide with a test substance which potentially associates with the polypeptide, under conditions which permit the association, and removing and/or detecting polypeptide associated with the test substance. Substance-polypeptide complexes, free substance, or non-complexed polypeptide may be assayed, or the activity of the polypeptide may be assayed. Conditions which permit the formation of substance-polypeptide complexes may be selected having regard to factors such as the nature and amounts of the substance and the polypeptide.

The substance-polypeptide complex, free substance or non-complexed polypeptide may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against a polypeptide of the invention or the substance, or labeled polypeptide, or a labeled substance may be utilized. The antibodies, polypeptide, or substances may be labeled with a detectable substance as described above.

A polypeptide of the invention, or the substance used in the method of the invention may be insolubilized. For example, a polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating an agent or test compound for its ability to modulate the biological activity of a polypeptide of the invention, by assaying for an agonist or antagonist (i.e. enhancer or inhibitor) of the association of the polypeptide with a substance which associates with the polypeptide. The basic method for evaluating if a compound is an agonist or antagonist of the association of a polypeptide of the invention and a substance that associates with the polypeptide, is to prepare a reaction mixture containing the polypeptide and the substance under conditions which permit the formation of substance-polypeptide complexes, in the presence of a test compound. The test compound may be initially added to the mixture, or may be added subsequent to the addition of the polypeptide and substance. Control reaction mixtures without the test compound or with a placebo are also prepared. The formation of complexes is detected and the formation of complexes in the control reaction but not in the reaction mixture indicates that the test compound interferes with the interaction of the polypeptide and substance. The reactions may be carried out in the liquid phase or the polypeptide, substance, or test compound may be immobilized as described herein. In an embodiment of the invention, the substance is a natural substrate or ligand of a polypeptide of the invention, or a structural or functional mimetic thereof.

It will be understood that the agonists and antagonists i.e. inhibitors and enhancers that can be assayed using the methods of the invention may act on one or more of the binding sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of a polypeptide of the invention with a substance which is capable of associating with or binding to the polypeptide. Thus, the invention may be used to assay for a compound that competes for the same binding site of a polypeptide of the invention.

In an embodiment, the invention provides a method of screening compounds to identify those which enhance (agonist) or block (antagonist) the action of polypeptides or polynucleotides of the invention, particularly those compounds that are bacteriostatic and/or bacteriocidal. The method of screening may involve high-throughput techniques. For example, to screen for agonists or antagoists, a synthetic reaction mix, a cellular compartment, such as a membrane, cell envelope or cell wall, or a preparation of any thereof, comprising a polypeptide of the invention and a labeled substrate or ligand of such polypeptide is incubated in the absence or the presence of a test compound that may be an agonist or antagonist. The ability of the test compound to agonize or antagonize the polypeptide is reflected in decreased binding of the labeled ligand or decreased production of product from such substrate. Molecules that bind gratuitously, i.e., without inducing the effects of a polypeptide of the invention are most likely to be good antagonists. Molecules that bind well and increase the rate of product production from substrate are agonists. Detection of the rate or level of production of product from substrate may be enhanced by using a reporter system. Reporter systems that may be useful in this regard include but are not limited to calorimetric labeled substrate converted into product, a reporter gene that is responsive to changes in polynucleotide or polypeptide activity, and binding assays known in the art.

Another example of an assay for antagonists is a competitive assay that combines a polypeptide of the invention and a potential antagonist with molecules that bind a polypeptide of the invention, a recombinant binding molecule, natural substrate or ligand, or substrate or ligand mimetic, under appropriate conditions for a competitive inhibition assay. The polypeptide can be labeled, such as by radioactivity or a calorimetric compound, such that the number of polypeptides bound to a binding molecule or converted to product can be determined accurately to assess the effectiveness of the potential antagonist.

Agents that modulate a polypeptide of the invention can be identified based on their ability to interfere with or enhance the activity of a polypeptide of the invention. Therefore, the invention provides a method for evaluating a compound for its ability to modulate the activity of a polypeptide of the invention comprising (a) reacting an acceptor molecule and a donor molecule for a polypeptide of the invention in the presence of a test compound; (b) measuring transfer of a sugar of the donor molecule to the acceptor molecule, and (c) carrying out steps (a) and (b) in the absence of the test compound to determine if the compound interferes with or enhances transfer of the sugar to the acceptor molecule by the polypeptide.

The acceptor molecule or donor molecule may be labeled with a detectable substance as described herein, and the interaction of the polypeptide of the invention with the acceptor molecule and donor molecule will give rise to a detectable change. The detectable change may be calorimetric, photometric, radiometric, potentiometric, etc. The activity of a polypeptide of the invention may also be determined using methods based on HPLC (Koenderman et al., FEBS Left. 222:42, 1987) or methods employing synthetic oligosaccharide acceptors attached to hydrophobic aglycones (Palcic et al Glycoconjugate 5:49, 1988; and Pierce et al, Biochem. Biophys. Res. Comm. 146: 679, 1987).

A polypeptide of the invention is reacted with the acceptor and donor molecules at a pH and temperature and in the presence of a metal cofactor, usually a divalent cation, effective for the protein to transfer the sugar of the donor molecule to the acceptor molecule, and where one of the components is labeled, to produce a detectable change. It is preferred to use a buffer with the acceptor and donor molecules to maintain the pH within the pH range effective for the proteins. The buffer, acceptor and donor molecules may be used as an assay composition. Other compounds such as EDTA and detergents may be added to the assay composition. The polypeptide may be obtained from natural sources or produced using recombinant methods as described herein.

The reagents suitable for applying the methods of the invention to evaluate compounds that modulate a polypeptide of the invention may be packaged into convenient kits providing the necessary materials packaged into suitable containers. The kits may also include suitable supports useful in performing the methods of the invention.

A substance that inhibits a polypeptide may be identified by treating a cell which expresses the polypeptide with a test substance, and analyzing the lipopolysaccharide structures on the cell. Lipopolysaccharide can be analyzed using the methods described herein. Cells that have not been treated with the substance or which do not express the polypeptide may be employed as controls.

Substances which inhibit transcription or translation of a hepT gene may be identified by transfecting a cell with an expression vector comprising a recombinant molecule of the invention, including a reporter gene, in the presence of a test substance and comparing the level of expression of a hepT, or the expression of the protein encoded by the reporter gene with a control cell transfected with the nucleic acid molecule in the absence of the substance. The method can be used to identify transcription and translation inhibitors of a hepT gene.

Compositions and Treatments

The polynucleotides and polypeptides of the invention and substances or compounds identified by the methods described herein, antibodies, and antisense nucleic acid molecules of the invention may be used to treat diseases. Examples of diseases that may be treated include diseases associated with organisms that contain a polypeptide or polynucleotide of the present invention. In an embodiment the organisms are from the Helicobacter family, and are particularly Helicobacter pylori species.

Helicobacter pylori infects the stomachs of over one-third of the world's population causing stomach cancer, ulcers, and gastritis (International Agency for Research on Cancer (1994) Schistosomes, Liver Flukes and Helicobacter Pylori (International Agency for Research on Cancer, Lyon, France; http://www.uicc.ch/ecp/ecp2904.htm). There is also a recognized cause-and-effect relationship between H. pylori and gastric adenocarcinoma, classifying the bacterium as a Group I (definite) carcinogen. Preferred agonists of the invention found using screens provided by the invention, particularly broad-spectrum antibiotics, will be useful in the treatment of H. pylori infection, and they should decrease the advent of H. pylori-induced cancers, such as gastrointestinal carcinoma. The agonists should also be useful in the treatment of gastric ulcers and gastritis.

Accordingly, the proteins, substances, antibodies, and compounds etc. may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences Remington's Pharmaceutical Sciences, Mack Publishing Company, Easton, Pa, USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

After pharmaceutical compositions have been prepared, they can be placed in an appropriate container and labeled for treatment of an indicated condition. For administration of a composition of the invention the labeling would include amount, frequency, and method of administration.

The compositions, substances, compounds etc. may be indicated as therapeutic agents either alone or in conjunction with other therapeutic agents or other forms of treatment (e.g. chemotherapy or radiotherapy). By way of example, they can be used in combination with anti-proliferative agents, antimicrobial agents, immunostimulatory agents, or anti-inflammatories. In particular, they can be used in combination with antibacterial agents. They can be administered concurrently, separately, or sequentially with other therapeutic agents or therapies.

Polynucleotides of the invention or any fragment thereof, or antisense sequences may be used for therapeutic purposes. Antisense to a polynucleotide encoding a polypeptide of the invention may be used in situations to block the synthesis of the polypeptide. In particular, cells may be transformed with sequences complementary to polynucleotides of the invention. Thus, antisense sequences may be used to modulate activity of a polypeptide of the invention, or to achieve regulation of gene function. Sense or antisense oligomers or larger fragments, can be designed from various locations along the coding or regulatory regions of sequences encoding a polypeptide of the invention.

Expression vectors may be derived from retroviruses, adenoviruses, herpes or vaccinia viruses or from various bacterial plasmids for delivery of nucleic acid sequences to the target organ, tissue, or cells. Vectors that express antisense nucleic acid sequences of hepT can be constructed using techniques well known to those skilled in the art (see for example, Sambrook et al. (supra)).

Genes encoding a HepT can be turned off by transforming a cell or tissue with expression vectors that express high levels of a polynucleotide of the invention. Such constructs may be used to introduce untranslatable sense or antisense sequences into a cell. Even if they do not integrate into the DNA, the vectors may continue to transcribe RNA molecules until all copies are disabled by endogenous nucleases.

Modification of gene expression may be achieved by designing antisense molecules, DNA, RNA, or Peptide nucleic acid (PNA), to the control regions of a HepT gene i.e. the promoters, enhancers, and introns. Preferably the antisense molecules are oligonucleotides derived from the transcription initiation site (e.g. between positions −10 and +10 from the start site). Inhibition can also be achieved by using triple-helix base-pairing techniques. Triple helix pairing causes inhibition of the ability of the double helix to open sufficiently for the binding of polymerases, transcription factors, or regulatory molecules (see Gee J. E. et al (1994) In: Huber, B. E. and B. I. Carr, Molecular and Immunologic Approaches, Futura Publishing Co., Mt Kisco, N.Y.). An antisense molecule may also be designed to block translation of mRNA by inhibiting binding of the transcript to the ribosomes.

Ribozymes may be used to catalyze the specific cleavage of RNA. Ribozyme action involves sequence-specific hybridization of the ribozyme molecule to complementary target RNA, followed by endonucleolytic cleavage. For example, hammerhead motif ribozyme molecules may be engineered that can specifically and efficiently catalyze endonucleolytic cleavage of sequences encoding a polypeptide of the invention.

Specific ribosome cleavage sites within any RNA target may be initially identified by scanning the target molecule for ribozyme cleavage sites which include the following sequences: GUA, GUU, and GUC. Short RNA sequences of between 15 and 20 ribonucleotides corresponding to the region of the cleavage site of the target gene may be evaluated for secondary structural features which may render the oligonucleotide inoperable. The suitability of candidate targets may be evaluated by testing accessibility to hybridization with complementary oligonucleotides using ribonuclease protection assays.

Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

Mutant Organisms

The invention provides novel mutants of Helicobacter bacteria, in particular mutants of H. pylori, having mutated (deactivated) hepT genes. In general, “mutated” refers to a sudden heritable change in the phenotype of an organism which can be spontaneous or induced by known mutagenic agents, including radiation and various chemicals.

Methods are known in the art that can be used to generate mutations to produce the mutant bacteria of the present invention. For example, the transposon, Tn10, can be used to produce chromosomal deletions in a wide variety of bacteria (Kleckner et al., J. Mol. Biol. 116:125-159, 1977; EPO Pub. No. 315,682; U.S. Pat. No. 5,387,744. Alternatively, methods may be used that involve introducing specific deletions in a hepT gene in an organism. A specific deletion in the selected gene can be generated by either of two general methods.

The first method generates a mutation in a gene isolated from a population of clones contained in a genomic DNA library using restriction enzymes and the second method generates the mutation in a gene of known sequence using PCR. Using the first method, the position of the gene on a vector is identified using transposon tagging and a restriction map of the recombinant DNA in the vector is generated. Information derived from the transposon tagging allows all or a portion of a gene to be excised from the vector using the known restriction enzyme sites.

The second method is based upon PCR. Divergent PCR primers are used to amplify the upstream and downstream regions flanking a specified segment of the hepT DNA to be deleted from the gene, generating a PCR product consisting of the cloning vector and upstream and downstream flanking nucleotide sequences (Innes et al. Eds., PCR Protocols, 1990, Academic Press, New York). In a variation of this method, PCR products are produced representing portions of the gene or flanking sequence, which are then joined together in a cloning vector.

Mutagenesis of a cloned hepT gene may also be carried out by insertion of a marker into an insertion site in the gene. For example, a kanamycin resistance marker may be ligated into an insertion site created in a hepT gene by reverse PCR (See Example 1).

The DNA containing the mutant gene can be introduced into the bacterial host by transformation using chemical means or electroporation, by recombinant phage infection, or by conjugation. In preferred embodiments the mutant gene is introduced into the chromosomes of the bacteria which can be accomplished using any of a number of methods well known in the art such as, for example, methods using temperature-sensitive replicons (Hamilton et al., J. Bacteriol. 171:4617-4622, 1989), linear transformation of recBC mutants (Jasin et al., J. Bacteriol. 159:783-786, 1984), or host restricted replicons known as suicide vectors (Miller et al., J. Bacteriol. 170:2575-2583, 1988). The particular method used is coupled with an appropriate counter selection method such as, for example, by using PCR, nucleic acid hybridization, or an immunological method.

The invention also provides modified LPS molecules from mutants of the invention. The modified LPS may be isolated from the mutant bacteria and at least partially purified using techniques well known to those skilled in the art. Preparations of at least 70%, particularly 80%, more particularly 90%, most particularly 95% pure LPS are preferred. The purity of an LPS preparation is expressed as the weight percentage of the total Helicobacter antigens present in the preparation. The purified LPS can be used as antigen either directly or after being conjugated to a suitable carrier protein.

Methods for Preparing Oligosaccharides

The invention relates to a method for preparing an oligosaccharide comprising contacting a reaction mixture comprising an activated donor molecule and an acceptor molecule in the presence of a polypeptide of the invention.

In an embodiment of the invention, the oligosaccharides are prepared on a carrier that is non-toxic to a mammal, in particular a lipid isoprenoid or polyisoprenoid alcohol. An example of a suitable carrier is dolichol phosphate. The oligosaccharide may be attached to a carrier via a labile bond allowing for chemical removal of the oligosaccharide from the lipid carrier. In the alternative, the oligosaccharide transferase may be used to transfer the oligosaccharide from a lipid carrier to a protein.

Vaccines

The mutant bacteria expressing the truncated LPS and the modified LPS isolated from such mutants are useful sources of antigens in vaccination against Helicobacter bacteria, in particular against H. pylori. Such vaccines are normally prepared from dead bacterial cells, using methods well known to those skilled in the art, and usually contain various auxiliary components, such as an appropriate adjuvant and a delivery system. A delivery system aiming at mucosal delivery is preferred. Preferably but not essentially, the antigenic preparation is administered orally to the host, but parenteral administration is also possible. Live vaccines based on H. pylori mutants may also be prepared, but would normally require an appropriate vector for mucosal delivery. Vaccines of the present invention are useful in preventing and reducing the number of H. pylori infections and indirectly in reducing the incidence of pathological conditions associated with such infections, in particular gastric cancer.

Another aspect of the invention relates to a method for inducing an immunological response in an individual, particularly a mammal which comprises inoculating the individual with an antigen (e.g. modified LPS) adequate to produce antibody and/or T cell immune response to protect said individual from infection, particularly bacterial infection and most particularly Helicobacter pylori infection. Also provided are methods whereby such immunological response slows bacterial replication.

A further aspect of the invention relates to an immunological composition which, when introduced into an individual capable of having induced within it an immunological response, induces an immunological response in such individual to Helicobacter wherein the composition comprises a modified LPS. The immunological response may be used therapeutically or prophylactically and may take the form of antibody immunity or cellular immunity such as that arising from CTL or CD4+T cells.

A modified LPS may be fused with a molecule which may not by itself produce antibodies, but is capable of stabilizing the modified LPS and producing an antigen which will have immunogenic and protective properties. Examples of such molecules are lipoprotein D from Hemophilus influenzae, glutathione-S-transferase (GST) or beta-galactosidase. Moreover, the molecule may act as an adjuvant in the sense of providing a generalized stimulation of the immune system.

The invention provides methods using the modified LPS in immunization experiments in animal models of infection with Helicobacter to identify epitopes able to provoke a prophylactic or therapeutic immune response. It is believed that this approach will allow for the subsequent preparation of monoclonal antibodies of particular value from the requisite organ of the animal successfully resisting or clearing infection for the development of prophylactic agents or therapeutic treatments of bacterial infection, particularly Helicobacter pylori infection, in mammals, particularly humans.

The modified LPS may be used as an antigen for vaccination of a host to produce specific antibodies which protect against invasion of bacteria, for example by preventing colonization.

The invention also includes a vaccine formulation which comprises a modified LPS of the invention together with a suitable carrier. The formulation is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation insotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier imnmediately prior to use. The vaccine formulation may also include adjuvant systems for enhancing the immunogenicity of the formulation, such as oil-in water systems and other systems known in the art The dosage will depend on the specific activity of the vaccine and can be readily determined by routine experimentation.

The following non-limiting examples are illustrative of the present invention:

### EXAMPLE 1

Functional analysis of waaF homologue (HP1191)

Complementation analysis was used to determine the function of the HP1191 from Helicobacter pylori strain 26695. The H. pylori HP1191 gene was amplified by PCR (primers: 5′GGGATCCCGGTCTTTAAACCCGCTCAACA3′ (SEQ ID NO: 5) and 5′GGGATCCCCGCTCTTCTCACGCCTTTAA3′ (SEQ ID NO: 6)) and cloned into pUC19 to obtain pHP1191 . WaaF mutant strain S. typhimurium 3789 was electroporated with this recombinant plasmid, and one of the resultant transformants selected for further study. SDS-PAGE was used to analyze LPS molecules produced by the relevant S. typhimurium strains. The LPS of the wild type strain formed the ladder like pattern indicative of the presence of the O antigen repeat unit whereas the LPS of the S. typhimurium waaF mutant appeared as a single fast migrating band. The migration pattern of this mutant was not affected by the presence of the plasmid vector. However, when the H. pylori gene HP1191 was present in trans in strain 3789, this S. typhimurium mutant synthesized an LPS which migrated in a pattern identical to that obtained with the LPS of the wild type strain. This confirmed the activity of HP1191 to be involved in catalyzing the addition of a second heptose molecule onto the heptose linked directly to KDO in the core.

To characterize the effect of the HP1191 mutations on LPS structure in H. pylori, proteinase K digested whole cell lysates from parent and mutant cells grown in broth were analyzed by SDS-PAGE. Silver staining revealed significant differences in the electrophoretic mobility of LPS isolated from parent and mutant cells of each strain examined. In all cases, LPS from mutant cells no longer produced S-type LPS but instead only a fast migrating rough type LPS was observed.

### EXAMPLE 2

Materials and methods

Cloning of the HP1191 and HP0279 genes A PCR product containing either the wild type H. pylori HP1191 or HP0279 gene was prepared using PwoI polymerase (Boehringer) and chromosomal DNA from H. pylori 26695. The primers used for amplification of HP1191 were: 5′GGGATCCCGGTCTTTAAACCCGCTCAACA3′ (SEQ ID NO: 5) and 5′GGGATCCCCGCTCTTCTCACGCCTTTAA3′ (SEQ ID NO: 6). The primers used for amplification of HP0279 were:5′ATCATTTAAAATACACGCTAG 3′ (SEQ ID NO:7) and 5′CCTACCGCTTCCCAATAG 3′ (SEQ ID NO: 8). The PCR fragments were ligated into pUC19 and used to transform E. coli DH5α. Both the HP1191 and HP0279 genes were cloned in this manner giving plasmids pHP1191 and p0279G1. (See SEQ ID NO: 1, 2, 3, and 4)

Mutational analysis of HP0279 Methods used for mutational analysis of HP0279 were carried out by the insertion of Kanamycin resistance marker from Campylobacter (Labigne-Roussel, et al, 1988). Briefly, a 1489 bp SmaI fragment of plasmid pIP1433 (Labigne-Roussel, et al., 1988), harboring a C. coli Kanamycin cassette, was inserted into the unique AflII site of the HP0279. The 5′ overhang produced by cutting the HP0279 gene with AflII was made blunt using T4 DNA polymerase to fill in the overhang. The resulting ligation mixture was used to transform E. coli DH5α and selection was carried out on media containing Kanamycin. Plasmid DNA was isolated from Kanamycin resistant colonies and the insertion of the Kanamycin cassette was confirmed by restriction digest analysis. One plasmid p279GM1 was used for transformation experiments into H. pylori. H. pylori strains were naturally transformed as previously described (Logan, et al, 2000).

Complementation of S. typhimurium WaaF/WaaC mutants Plasmids pHP1191 and p0279G1 were used to electroporate either the waaF (S. typhimurium 3789) or waaC (S. typhimurium 1377) mutant strain of S. typhimurium and respective parent strains S. typhimurium 3770 and 1355 (all S. typhimurium strains from the Salmonella Genetic Stock Centre, University of Calgary). The desired recombinant strains were selected with ampicillin. All standard methods of DNA manipulation were performed according to Sambrook et al (1989). The LPS profiles of the recombinants were analyzed by SDS-PAGE using methods previously described (Logan and Trust, 1984).

Results and Discussion

Transformation of H. pylori with mutant plasmid p279GM1 failed to produce transformants. Several unsuccessful attempts. were made to recover HP0279 mutant strains of H. pylori suggesting that mutations to this gene may be lethal.

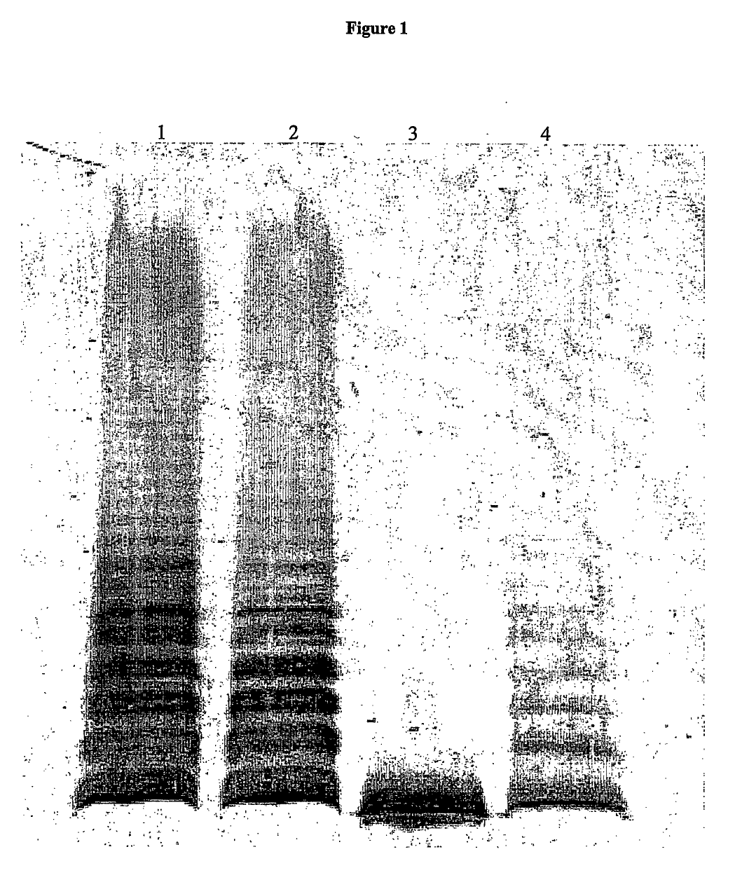

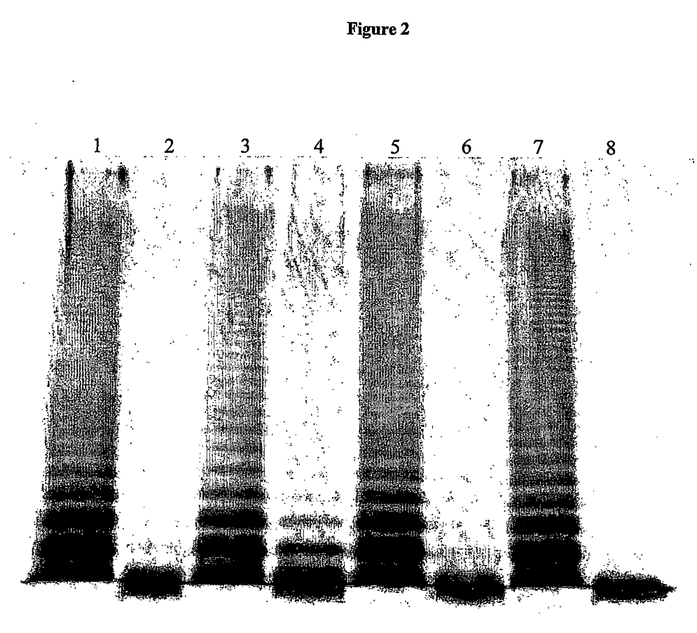

Complementation analysis was used to determine whether the similarity between WaaF and WaaC proteins of Helicobacter pylori and Salmonella typhimurium would allow the Helicobacter enzymes to function in the S. typhimurium host and if the proteins displayed similar specificities. The H. pylori waaF and waaC genes were cloned into pUC19 to obtain pHP1191 and p0279G1 respectively. The waaF mutant strain S. typhimurium 3789 and waaC mutant strain S. typhimurium 1377 and respective parent strains 3770 (waaF) and 1355 (waaC) were electroporated with the recombinant plasmids, and resultant tansformants were selected for further study. SDS-PAGE was used to analyze LPS molecules present in proteinase K treated whole cell lysates. (FIG. 1) The LPS of wild type S. typhimurium 3770 (FIG. 1, lane 1) and 1355 (FIG. 2 lane 1) both displayed a ladder like pattern indicative of long O-chain polysaccharide attached to core LPS. The LPS of both the waaC and waaF mutants appeared as a single fast migrating band typical of rough, core LPS only. The appearance of this mutant LPS was unaffected by the presence of plasmid (FIG. 1A lane 1,2, FIG. 2 lane 1,3, 5,7). However, when the wild type H. pylori waaF gene was present in trans in strain 3789 and the waaC gene in strain 1377, these mutant strains of S. typhimurium produced an LPS which migrated in a pattern identical to that of wild type LPS (FIG. 1 lane 4, FIG. 2 lane 4). Reciprocal experiments with H. pylori waaF gene in S. typhimurium waaC mutant and H. pylori waaC gene in the S. typhimurium waaF mutant were unsuccessful in restoring the wild-type LPS pattern (FIG. 2 lane 6,8). It appears therefore that the specificity of the waaC and waaF proteins are maintained in H pylori.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

### REFERENCES

1. Alm, R.A., L-S.L. Ling, D. T. Moir, B. L. King, E. D. Brown, P. C. Doig, D. R. Smith, B. Noonan, B. C. Guild, B. L. deJonge, G. Carmel, P. J. Tummino, A. Caruso, M. Uria-Nickelsen, D. M. Mills, C. Ives,. R. Gibson, D. Merberg, S. D. Mills, Q. Jiang, D. E. Taylor, G. F. Vovis, and T. J. Trust 1999. Genomic-sequence comparison of two unrelated isolates of the human gastric pathogen Helicobacter pylori. Nature 397(6715):176-180.

2. Labigne-Roussel, A., P. Courroux, and L. Tompkins. 1988. Gene disruption and replacement as a feasible approach for mutagenesis of Campylobacter jejuni. J. Bacteriol. 170(4): 1704-1708.

3. Logan, S. M., and T. J. Trust. 1984. Structural and antigenic heterogeneity of lipopolysaccharides of Campylobacter jejuni and Campylobacter coli. Infect. Immun. 45:210-216.

4. Logan, S. M., J. W. Conlon, M. A. Monteiro, W. W. Wakarchuk, and E. Altman. 2000. Functional genomics of Helicobacter pylori: Identification of a β-1,4 galactosyltransferase and generation of mutants with altered lipopolysaccharide. Mol. Microb. 35(5): 1156-1167.

5. Sambrook, J., E. F. Fritsch, and T. Maiatis. 1989. Molecular Cloning: A Laboratory Manual, 2^{nd} edn. Cold Spring Harbor, New York. Cold Spring Harbor Laboratory Press

6. Tomb J-F., O. White, A. R. Kerlavage, R. A. Clayton, G. G. Sutton, R. D. Fleischmann, K. A. Ketchum, H. P. Klenk, S. Gill, B. A. Dougherty, K. Nelson, J. Quackenbush, L. Zhou, E. F. Kirkness, S. Peterson, B. Loftus, D. Richarson, R. Dodson, H. G. Khalak, A. Glodek, K. McKenney, L. M. Fitzegerald, N. Lee, M. D. Adams, E. K. Hickey, D. E. Berg, J. D. Gocayne, T. R. Utterback, J. D. Peterson, J. M. Kelley, M. D. Cotton, J. M. Weidman, C. Fujii, C. Bowman, L. Watthey, E. Wallin, W. S. Hayes, M. Borodovsky, P. D. Karp, H. O. Smith, C. M. Fraser and J. C. Venter. 1997. The complete genome sequence of the gastric pathogen Helicobacter pylori. Nature 388:539-547.

We claim:

1. An isolated heptosyltransferase (hepT) polynucleotide of at least 30 nucleotides which hybridizes to SEQ ID NO. 1 or 3 or the complement of SEQ ID NO. 1 or 3 under stringent hybridization conditions.

2. An isolated hepT polynucleotide which comprises:
(a) a polynucleotide encoding a polypeptide having substantial sequence identity, preferably at least 50%, more preferably at least 70% sequence identity, with an amino acid sequence of SEQ. ID. NO. 2 or 4;
(b) polynucleotides complementary to (a);
(c) polynucleotides differing from any of the polynucleotides of (a) or (b) in codon sequences due to the degeneracy of the genetic code;
(d) a polynucleotide comprising at least 10, 15, or 18, preferably at least 20 nucleotides and capable of hybridizing under stringent conditions to a polynucleotide of SEQ. ID. NO. 1 or 3 or to a degenerate form thereof;
(e) a polynucleotide encoding an allelic or species variation of a polypeptide comprising an amino acid sequence of SEQ. ID. NO. 2 or 4; or
(f) a fragment, or allelic or species variation of (a), (b) or (c).

3. An isolated polynucleotide as claimed in claim 2 which comprises:
(a) a polynucleotide having substantial sequence identity, preferably at least 50%, more preferably at least 70% sequence identity with a sequence of SEQ. ID. NO. 1 or 3;
(b) polynucleotides complementary to (a), preferably complementary to a full sequence of SEQ. ID. NO. 1 or 3;
(c) polynucleotides differing from any of the polynucleotides of (a) to (b) in codon sequences due to the degeneracy of the genetic code; or
(d) a fragment, or allelic or species variation of (a), (b) or (c).

4. An isolated polynucleotide which encodes a polypeptide which binds an antibody of a HepT derived from Helicobacter pylori.

5. A vector comprising a polynucleotide of claim 1, 2, 3 or 4.

6. A host cell comprising a polynucleotide of any preceding claim.

7. An isolated heptosyltransferase polypeptide comprising an amino acid sequence of SEQ. ID. NO. 2, or 4.

8. An isolated polypeptide having at least 70% amino acid sequence identity to an amino acid sequence of SEQ. ID. NO. 2 or 4.

9. A method for preparing a HepT polypeptide comprising an amino acid sequence of SEQ. ID. NO. 2 or 4 comprising:
(a) transferring a vector as claimed in claim 5 into a host cell;
(b) selecting transformed host cells from untransformed host cells;
(c) culturing a selected transformed host cell under conditions which allow expression of the polypeptide; and
(d) isolating the polypeptide.

10. A recombinant polypeptide prepared in accordance with the method of claim 9.

11. An antibody having specificity against an epitope of a polypeptide as claimed in claim 7 or 8.

12. An antibody as claimed in claim 11 labeled with a detectable substance and used to detect the polypeptide in biological samples, tissues, and cells.

13. A probe comprising a sequence encoding a polypeptide as claimed in claim 7 or 8, or a part thereof.

14. A method of diagnosing and monitoring diseases by determining the presence of a polynucleotide or a polypeptide as claimed in any preceding claim.

15. A method for identifying a substance which associates with a polypeptide as claimed in claim 7 or 8 comprising
(a) reacting the polypeptide with at least one substance which potentially can associate with the polypeptide, under conditions which permit the association between the substance and polypeptide, and
(b) removing or detecting polypeptide associated with the substance, wherein detection of associated polypeptide and substance indicates the substance associates with the polypeptide.

16. A method as claimed in claim 15 wherein association of the polypeptide with the substance is detected by assaying for substance-polypeptide complexes, for free substance, for non-complexed polypeptide, for enzymatic activity of the polypeptide, or for activation of the polypeptide.

17. A method for identifying compounds which bind to or otherwise interact with and inhibit or activate an activity of a polypeptide or polynucleotide as claimed in any preceding claim comprising:
(a) contacting a polypeptide or polynucleotide as claimed in any preceding claim with a test compound under conditions to permit binding to or other interaction between the test compound and the polypeptide or polynucleotide to assess the binding to or other interaction with the test compound, wherein the binding or interaction is associated with a second component capable of providing a detectable signal in response to the binding or interaction of the polypeptide or polynucleotide with the test compound; and
(c) determining whether the test compound binds to or interacts with and activates or inhibits an activity of the polypeptide or polynucleotide by detecting the presence or absence of a signal generated from the binding or interaction of the test compound with the polypeptide or polynucleotide.

18. A method for evaluating a test compound for its ability to modulate the activity of a polypeptide as claimed in any preceding claim comprising:
(a) reacting an acceptor molecule and a donor molecule for the polypeptide in the presence of a test compound;
(b) measuring transfer of a sugar of the donor molecule to the acceptor molecule, and
(c) carrying out steps (a) and (b) in the absence of the test compound to determine if the compound interferes with or enhances transfer of the sugar of the donor molecule to the acceptor molecule by the polypeptide.

19. A method for formulating a pharmaceutical composition comprising:
(a) conducting therapeutic profiling of test compounds identified in accordance with a method as claimed in claim 17 or 18, or further analogs thereof, for efficacy and toxicity in animals; and
(b) formulating a pharmaceutical composition including one or more test compounds identified in step (a) as having an acceptable therapeutic profile.

20. A method as claimed in claim 19 further comprising establishing a distribution system for distributing the pharmaceutical composition for sale, and optionally establishing a sales group for marketing the pharmaceutical composition.

21. A method of conducting a target discovery business comprising:
(a) providing a method as claimed in claim 17 or 18 for identifying test compounds that bind to or interact with and activate or inhibit or modulate an activity of the polypeptide or polynucleotide;
(b) optionally conducting therapeutic profiling of test compounds identified in (a) for efficacy and toxicity in animals; and
(c) licensing to a third party the rights for further drug development and/or sales for test compounds identified in step (a), or analogs thereof.

22. A method for detecting a polynucleotide encoding a polypeptide comprising an amino acid sequence of SEQ. ID. NO. 2 or 4 in a biological sample comprising the steps of:
(a) hybridizing a polynucleotide as claimed in any preceding claim to nucleic acids of the biological sample, thereby forming a hybridization complex; and
(b) detecting the hybridization complex wherein the presence of the hybridization complex correlates with the presence of a nucleic acid encoding the polypeptide in the biological sample.

23. A method as claimed in claim 22 wherein nucleic acids of the biological sample are amplified by the polymerase chain reaction prior to the hybridizing step.

24. A method for treating a disease comprising administering an effective amount of an antibody as claimed in claim 11 or a substance or compound identified in accordance with a method claimed in claim 15, 17, or 18.

25. A composition comprising one or more of a polynucleotide as claimed in any preceding claim or a polypeptide claimed in claim 7 or 8, and a pharmaceutically acceptable carrier, excipient or diluent.

26. A method for preparing an oligosaccharide comprising contacting a reaction mixture comprising an activated donor molecule, and an acceptor in the presence of a polypeptide as claimed in claim 7 or 8.

27. A mutant Helicobacter pylori having one or more inactivating mutations in a hepT gene which render the Helicobacter pylori avirulent.

28. A method for preparing an immunogenic composition comprising mixing a mutant Helicobacter pylori according to claim 27 with a pharmaceutically acceptable carrier.

29. An immunogenic composition for use in a human comprising a live avirulent derivative of Helicobacter pylori having one or more inactivating mutations in a hepT gene which render the Helicobacter pylori avirulent.

30. A mutant strain of H. pylori, said mutant strain having a deactivated hepT gene

31. A vaccine composition comprising an antigen derived from a mutant strain of H. pylori according to claim 27 or 30.

32. A vaccine composition according to claim 31, wherein the antigen is an at least partially purified lipopolysaccharide.

33. A vaccine composition according to claim 32, wherein the antigen is conjugated to a protein.

34. A live attenuated vaccine composition comprising a mutant strain of H. pylori according to claim 27 or 30.

35. A reaction mixture for an enzymatic synthesis of a Helicobacter lipopolysaccharide or a portion thereof, the mixture comprising an isolated polypeptide as claimed in claim 7 or 8.

36. A reaction mixture according to claim 35, wherein the bacterial lipopolysaccharide is a mimic of a Helicobacter lipopolysaccharide.
